(19) United States
(12) Patent Application Publication
(10) Pub. No.: US 2023/0040158 A1
(43) Pub. Date: Feb. 9, 2023

(54) MOLECULAR DATA STORAGE SYSTEMS AND METHODS

(71) Applicant: TECHNION RESEARCH AND DEVELOPMENT FOUNDATION LTD., Haifa (IL)

(72) Inventors: Leon ANAVY, Haifa (IL); Zohar YAKHINI, Ramat Hasharon (IL); Roee AMIT, Haifa (IL)

(21) Appl. No.: 17/780,404

(22) PCT Filed: Nov. 27, 2019
(86) PCT No.: PCT/IL2019/051300

§ 371 Date: May 26, 2022

(51) Int. Cl. G06N 3/12; G16B 30/00

(52) U.S. Cl. CPC G06N 3/123; G16B 30/00

(57) ABSTRACT

A molecular data storage system is presented for encoding data-block(s). The system includes one or more populations of molecular sequences, each population encoding a respective one of the data-blocks. Each molecular sequence comprises a data encoding section comprising a sequence of similar predetermined length N of short k-mers, whereby in each population the data encoding sections of all molecular sequences have the similar predetermined length N. The short k-mers serve as data encoding building blocks of the data encoding sections, whereby valid short k-mers serving as data encoding building blocks form a subset of a building-block-set consisting of a number Z of different preselected short k-mers each presenting a unique combination of a number k of bases of a preselected set of bases, characterized in that all the Z types of short k-mers in said building-block-set have a similar predetermined size k≥2 (plurality) of bases. The data encoding sections collectively encode a sequence of encoded alphabet letters S=(π¹, π², . . . , πⁿ . . . , π^{N-1}, π^{N}). Each valid encoded alphabet letter πⁿ at location n of the sequence S of alphabet letters is characterized by occurrence of a predetermined plurality of different types of short k-mers of the building-block-set in a corresponding location n along the data encoding sections of the plurality of molecular sequences of said population.

### TECHNOLOGICAL FIELD AND BACKGROUND

The present invention is in the field of data storage technologies and is particularly related to molecular data storage systems and methods, such as DNA based data storage.

In recent years, various DNA based data storage systems have been developed. Such systems are advantageous because of their remarkable data density and long-term stability of DNA. The first demonstrations of DNA based data storage, on a megabyte scale, were revealed in 2012 in two independent studies^{1,2}. In a recent work, the Shannon information capacity of DNA was demonstrated, using fountain code error correction, to be ˜1.57 bit per synthesized position³.

### BACKGROUND ART

References considered to be relevant as background to the presently disclosed subject matter are listed below:
1. Church G M, Gao Y, Kosuri S. Next-generation digital information storage in DNA. Science. 2012; 337 (6102):1628. doi:10.1126/science.1226355
2. Goldman N, Bertone P, Chen S, Birney E. Towards practical, high-capacity, low-maintenance information storage in synthesized DNA. Nature. 2013; 494(7435):77-80. doi:10.1038/nature11875
3. Erlich Y, Zielinski D. DNA Fountain enables a robust and efficient storage architecture. Science (80-). 2017; 355(6328):950-954. doi:10.1126/science.aaj2038
4. Mcginn S, Gut I G. DNA sequencing—spanning the generations. N Biotechnol. 2013; 30(4):366-372. doi:10.1016/j.nbt.2012.11.012
5. LeProust E M, Peck B J, Spirin K, . . . Caruthers M H. Synthesis of high-quality libraries of long (150mer) oligonucleotides by a novel depurination controlled process. Nucleic Acids Res. 2010; 38(8):2522-2540. doi:10.1093/nar/gkq163
6. Jimenez-Sanchez A. Dna Computer Code Based on Expanded Genetic Alphabet. Eur J Comput Sci Inf Technol. 2014; 2(4): 8-20. doi:10.13140/2.1.3305.4408
7. Ouchi M, Badi N, Lutz J F, Sawamoto M. Single-chain technology using discrete synthetic macromolecules. Nat Chem. 2011; 3(12):917-924. doi:10.1038/nchem.1175
8. Lutz J F, Ouchi M, Liu D R, Sawamoto M. Sequence-controlled polymers. Science (80-). 2013; 341(6146). doi:10.1126/science.1238149
9. Organick L, Ang S D, Chen Y-J, . . . Strauss K. Random access in large-scale DNA data storage. Nat Biotechnol. 2018; 36(3):242-248. doi:10.1038/nbt.4079
10. Loose M, Malla S, Stout M. Real-time selective sequencing using nanopore technology. Nat Methods. 2016; 13 (9):751-754. doi:10.1038/nmeth.3930
11. Ferrante M, Saltalamacchia M. The Coupon Collector's Problem. 2014; 35(2):pp. www.mat.uab.cat/matmat. Accessed Dec. 3, 2018.
12. Anavy L, Vaknin I, Atar O, Amit R, Yakhini Z. Data storage in DNA with fewer synthesis cycles using composite DNA letters. Nat Biotechnol. 2019; 37(10):1237-1237. doi:10.1038/s41587-019-0281-1

Acknowledgement of the above references herein is not to be inferred as meaning that these are in any way relevant to the patentability of the presently disclosed subject matter.

### GENERAL DESCRIPTION

There is a need in the art for a novel approach to molecular based data storage techniques, e.g. DNA-based storage systems, with improved data storage capacity/density.

Indeed, current DNA synthesis and sequencing technologies process large numbers of nominally identical molecules in parallel^{4,5}, which leads to significant information redundancy that is inherent in conventional DNA based storage schemes.

The present invention provides a novel technique for molecular based data storage technique which one the one hand exploits and reduces the inherent data redundancy that characterizes molecular data storage system data redundancy and improves the data density of the data storage, and on the other hand improves the data storage resilience to various data errors, such as synthesizing errors occurring during writing of the data, sequencing errors occurring during synthesizing of the data, and also degradation errors introduced during time. This is achieved by various aspects and embodiments of the present invention as described in details in the following.

According to one broad aspect of the present invention there is provided a molecular data storage system for encoding one or more data-blocks, which comprises one or more populations of molecular sequences, each population of molecular sequences encoding a respective data-block of the one or more data-blocks. Each molecular sequence of the molecular sequences of the population comprises a data encoding section comprising a sequence of similar predetermined length N of short k-mers, whereby in each population the data encoding sections of all molecular sequences have the similar predetermined length N. The short k-mers serve as data encoding building blocks of the data encoding sections, whereby valid short k-mers serving as data encoding building blocks form a subset of a building-block-set consisting of a number Z of different preselected short k-mers each presenting a unique combination of a number k of bases of a preselected set of bases, characterized in that all the Z types of short k-mers in said building-block-set have a similar predetermined size k≥2 (plurality) of bases. The data encoding sections of the molecular sequences of the population collectively encode a sequence of encoded alphabet letters S=(π¹, π², . . . , πⁿ . . . , π^{N-1}, π^{N}). Each valid encoded alphabet letter e at location n of the sequence S of alphabet letters is characterized by occurrence of a predetermined plurality of different types of short k-mers of the building-block-set in a corresponding location n along the data encoding sections of the plurality of molecular sequences of said population.

In some embodiments, each valid encoded alphabet letter it at location n of the sequence S of alphabet letters is further characterized by occurrence of a predetermined exact number Y of the different types of short k-mers of the building-block-set in said corresponding location n in the data encoding sections, said predetermined exact number Y being the same for all the valid encoded alphabet letters; thereby enabling robust and efficient sequencing protocol by validating a letter encoded at said location n based on equality between said predetermined exact number Y and an actual number of Y′ of different types of short k-mers observed at said corresponding location n of said data encoding sections.

In some embodiments, all the different types of preselected short k-mers in said building-block-set have a similar predetermined size k≤20 of bases, thereby facilitating production scale data storage via molecular synthesis and low physical density. Preferably, the similar predetermined size of the bases is k≤10.

In some embodiments, the Z different types of short k-mers in said building-block-set are characterized in that a hamming distance between each short k-mer in said building-block-set and any other short k-mer in said building-block-set is greater or equal to a certain first H1 threshold of minimal hamming distance, whereby said first threshold satisfies H1≥2, thereby enabling robust reading with error correction. Preferably, the certain first H1 threshold of minimal hamming distance satisfies H1≥4.

In some embodiments, each valid encoded alphabet letter πn of the sequence S=(π¹, π², . . . , πⁿ . . . , π^{N-1}, π^{N}) belongs to a set of predefined alphabet letters Σ≡{σₘ}|_{m=1 to M} defined as binary occurrence vectors over the space spanned by said Z different types of short k-mer building blocks. For example, the set of predefined alphabet letters Σ≡{σₘ}|_{m=1 to M} consists only of binary occurrence vectors of equal weight. In some examples, the set of predefined alphabet letters Σ≡{σₘ}|_{m=1 to M} consists only of binary occurrence vectors of said space having hamming distances between them greater or equal to a certain second threshold H2 of minimal hamming distance wherein said second threshold H2 of minimal hamming distance is at least (H2≥2). The second threshold H2 of minimal hamming distance may be at least (H2≥4).

In some embodiments, the different types of short k-mers in said building-block-set are composed of molecular bases of any one of the following base-sets: [A, C, G, T], [A, C, G, U].

In some embodiments, the size Sz of said base-set is 4.

In some embodiments, each molecular sequence of the molecular sequences of the population includes a population identification section comprising an identifying sequence of molecular bases indicative of the population with which said molecular sequence is associated; and wherein said identifying sequence is different in molecular sequences associated with different ones of said plurality of populations.

The configuration may be such that the molecular bases included in said population identification section are bases of the same preselected set of bases by which said building-blocks are constructed. For example, the molecular bases included in said population identification section are bases of the same preselected set of bases by which said building-blocks are constructed. The population identification section may comprise an identifying sequence of said building-blocks.

A difference between the identifying sequences that are used in the population identification sections of different respective populations may exceed a predetermined threshold measured by a certain predetermined distance metric of strings, such as an edit distance metric between strings.

The molecular sequences of one or more of said plurality of populations may be contained together in a common region. The molecular sequences associated with the same population can be exclusively selected by utilizing binding molecules configured and operable for selectively binding to the population identification section of the molecular sequences associated with said same population.

In some embodiments, the system comprises a structure defining a plurality of distinct regions at which the molecular sequences of different respective populations reside. The molecular sequences of the different respective populations may reside exclusively and respectively at said distinct regions.

According to another broad aspect, the invention provides a method for reading data stored in a molecular data storage system. The method comprises:

(i) providing a molecular data storage system comprising a population of molecular sequences defining a data-block of the system;

(ii) applying sequencing to the population of molecular sequences and determining, per each location n of 1 to N locations in the data encoding sections of sequenced molecular sequences/of said population, an observed binary vector Xⁿ of dimension Z, whereby each binary component indexed z of 1 to Z binary components of the observed binary vector Xⁿ is indicative of whether a corresponding building block E^{z} of a building-block-set {E^{z}}|_{z=1 to Z} was found at the location n corresponding to the index of said binary vector Xⁿ along any of the sequenced molecular sequences of said population; wherein said molecular sequences of the population of said molecular data storage system comprise respective data encoding sections of similar predetermined length N of short k-mers serving as data encoding building blocks and forming a building-block-set {E^{z}}|_{z=1 to Z} consisting of a number Z of different preselected short k-mers by which data of the data-block is encoded, whereby each data encoding building block is a unique combination of a number k of bases of a preselected set of bases and wherein all the Z types of short k-mers in said building-block-set have a similar predetermined size k≥2 of bases; and

(iii) determining encoded alphabet letters πⁿ of a sequence S=(π¹, π², . . . , πⁿ . . . , π^{N-1}, π^{N}) encoded by said n=1 to N locations by associating each observed binary vector Xⁿ of each of said n=1 to N locations, to one of alphabet letters {σₘ} of a predetermined alphabet Σ≡{σₘ}|_{m=1 to M}; whereby each letter σₘ of the alphabet Σ is defined by a binary occurrence vector of size Z indicative of an occurrence of building blocks of said building-block-set {E^{z}} in the letter; said associating comprises mapping the observed binary vector Xⁿ at each location n to one of the letters {σₘ}|_{m=1 to M} of the alphabet Σ by determining a match between the observed binary vector Xⁿ and the binary vector definition of the letters.

In some embodiments, the Z different types of short k-mers in said building-block-set are characterized in that a hamming distance between each short k-mer in said building-block-set and any other short k-mer in said building-block-set is greater or equal to a certain first H1 threshold of minimal hamming distance, whereby said first threshold satisfies H1≥2. Said determining of the observed binary vector Xⁿ of dimension Z associated with location n in the data encoding sections comprises ignoring sequenced short k-mer found at said location in one or more of the data encoding sections which does not belong to the building block set.

In some embodiments, said predefined alphabet Σ≡{σₘ}|_{m=1 to M} consists only of binary vectors with hamming distances between them being greater or equal to a certain second threshold H2≥2 of minimal hamming distance, thereby providing that in case said match between the observed binary vector Xⁿ and said vector of definition one of the letters {σₘ}|_{m=1 to M} of the alphabet Σ is determined, said match being indicative of validity of the reading of the encoded letter πn from the locations n in said data encoding sections of sequenced molecular sequences.

The sequencing process may be conducted to a predetermined sequencing depth.

In some embodiments, each letter σₘ in the alphabet of letters Σ≡{σₘ}|_{m=1 to M} may be defined by occurrence of a predetermined exact number Y of the different types of short k-mers of said building-block-set {E^{z}}, said predetermined exact number Y being the same for all the valid encoded alphabet letters its. A stopping condition of said sequencing is that per each location n of said 1 to N locations of the data encoding sections at least said exact number Y of different types of short k-mers belonging to said building-block-set {E^{z}} is found. The sequencing may be carried out at least until said stopping condition is fulfilled or until a predetermined maximal sequencing depth.

In some embodiments, each letter σₘ in the alphabet of letters Σ≡{σₘ}|_{m=1 to M}, may be defined by occurrence of a predetermined and constant exact number Y of the different types of short k-mers of said building-block-set {E^{z}}, said predetermined exact number Y being the same for all the alphabet letters; and a data reading validation/correction operation is performed, by selectively performing, for each location n of said 1 to N locations of the data encoding sections at which a respective letter expected to be encoded, the following operations:

In case a weight Y′ of said observed binary vector Xⁿ is equal to said exact number Y, the encoded alphabet letters πⁿ at the location n is determined by mapping the observed binary vector Xⁿ to one of the alphabet letters {σₘ}|_{m=1 to M} based on a match between the observed binary vector Xⁿ and a binary vector representation of said one alphabet letter.

In case a weight Y′ of the observed binary vector Xⁿ is larger than said exact number Y, an excess Y′−Y of different types of building blocks is found at the locations n of the data encoding sections; and statistical significances is computed for each of the Y′ different types of building blocks found at the location n based on a number of times each of said Y′ types of building blocks is sequenced from the locations n. To this end, in case statistical significance of Y′−Y types of said Y′ building blocks are below a predetermined statistical significance threshold ST, the following is carried out: determining that said excess Y′−Y types of building blocks are the Y′−Y types of building blocks for which the statistical significance is below the threshold ST and amending said observed binary vector Xⁿ accordingly to obtain an amended observed binary vector X′ⁿ of weight Y; and determining said encoded alphabet letters πⁿ at the location n by mapping the amended observed binary vector X′ⁿ to one of the alphabet letters {σₘ}|_{m=1 to M} based on a match between the amended observed binary vector X′ⁿ and a binary vector representation of said one alphabet letter. In case there are less than Y′−Y types of said Y′ building blocks whose statistical significances are below the predetermined statistical significance threshold ST, it is determined that the observed binary vector Xⁿ may not be mapped to any one of the alphabet letters {σₘ}|_{m=1 to M} and thereby the encoded alphabet letters πⁿ at the location n is invalid.

In case a weight Y′ of said observed binary vector Xⁿ is less than said exact number parameter Y, it is determined that the observed binary vector Xⁿ may not be mapped to any one of the alphabet letters {σₘ}|_{m=1 to M} and thereby the encoded alphabet letter πn at the location n is invalid.

According to yet another broad aspect of the invention, it provides a data reader system adapted to read data stored in a molecular data storage system, and being configured and operable for implementing the above-described method. The data reader system comprises:

a) a sequencing control module configured and operable for connecting to a sequencing system for operating the sequencing system to perform the above-described operations (i) and (ii) to thereby sequence a population of molecular sequences of the data storage system; and

b) a data inference processing module configured and operable for carrying out the above-described operation (iii) to determine a sequence S={πⁿ}|_{n=1 to N} of encoded letters of the alphabet Σ being inferred from the population of molecular sequences.

The sequencing control module may be adapted to implement the above-described method and operate the sequencing system at least to a predetermined maximal sequencing depth.

In some embodiments described above, each letter σₘ in the alphabet of letters Σ≡{σₘ}|_{m=1 to M} is defined by occurrence of a predetermined exact and constant number Y of the different types of short k-mers of said building-block-set {E^{z}}, said predetermined exact number Y being the same for all the alphabet letters. The sequencing control module may be adapted to perform the above-described method and to operate the sequencing system at least until the stopping condition is fulfilled or until a predetermined maximal sequencing depth. The data inference processing module may be configured and operable to carry out a data reading validation/correction operation as described above.

The invention in its yet further broad aspect provides a method for fabricating a molecular data storage system. The method comprises:

a. providing a support substrate having one or more spatially separated regions at which one or more respective populations of molecular sequences can be synthesized;

b. providing one or more blocks of data to be respectively encoded by the one or more respective populations of molecular sequences which are to be synthesized at said one or more spatially separated regions respectively; wherein said one or more blocks of data are coded by a sequence of letters S={πⁿ}|_{n=1 to N} of an alphabet Σ≡{σₘ}|_{m=1 to M};

C. per each block of data, synthesizing a corresponding population of molecular sequences at a respective region of said one or more regions;

wherein the letters {σₘ}|_{m=1 to M} of the alphabet Σ are represented as binary occurrence vectors defined over a space spanned by Z different types of short k-mers of length k>1, which serve as data encoding molecular building blocks {Eₙ}|_{n=1 to Z} of the molecular data storage system; and wherein said synthesizing of the population of molecular sequences at the respective region includes synthesizing the sequences of letters S={πⁿ}|_{n=1 to N} of said block of data by selectively depositing, per each letter 70, all and only the data encoding building blocks indicated to be occurring by the binary vector representing the letter its.

In some embodiments, the depositing comprises:

(i) providing and placing said data encoding molecular building blocks indicated to be occurring by the binary vector representing the letter πn and placing them at said respective region to thereby enable their binding to molecules at said region; whereby the provided data encoding molecular building blocks are chemically “blocked” from one end thereof to prevent their binding to one another;

(ii) washing said region to remove un-bonded data encoding molecular building-blocks; and

(iii) applying un-blocking treatment to “un-block” the data encoding molecular building-blocks that are bounded to molecules at said region.

The region of the support plate may comprise cleavable molecules adapted to bind with said data encoding molecular building-blocks, such that deposition of the basic molecular building-blocks of the first letter π¹ being encoded, are bounded to said cleavable molecules. The method may comprise harvesting said population of molecules from said respective region by cleaving said cleavable molecules.

In some embodiments, the synthesizing of the population of molecule sequences comprises synthesizing similar population identification segments, in all molecule sequences of said population; whereby the population identification segment of each molecular sequence is indicative of the population with which the molecular sequence is associated and is different in molecular sequences of different populations.

According to yet further broad aspect of the invention, there is provided a molecular data storage fabrication system adapted to fabricate a molecular data storage structure. The molecular data storage fabrication system is configured and operable for implementing the above-described method for fabricating a molecular data storage system, and comprises:

a container module comprising a plurality of containers including at least Z containers adapted for respectively containing Z different types of short k-mers of length k>1, being respectively data encoding molecular building-blocks serving respectively as data encoding molecular building blocks {Eₙ}|_{n=1 to Z} of the molecular data storage system;

a fabrication head fluidly connected to said Z containers and configured and operable for controlled deposition of basic molecular building-blocks contained in a one or more selected containers out of said Z containers; and

a control unit configured and operable to operate the fabrication head for implementing operations (b) and (c) of the above-described method by carrying out the following:
providing at least one block of data to be encoded by synthesizing a respective population of molecular sequences encoding said block of data, on a region designated for carrying said population; wherein said at least one block of data is coded by a sequence of letters S={πⁿ}|_{n=1 to N} of an alphabet Σ≡{σₘ}|_{m=1 to M}; and wherein the letters {σₘ}|_{m=1 to M} of the alphabet Σ are represented as binary vectors (occurrence vectors) defined over a space spanned by Z different types of said data encoding molecular building blocks {Eₙ}|_{n=1 to Z}; and
synthesizing the population of molecular sequences encoding said block of data at the designated region, by operating said fabrication head, at said designated region to sequentially synthesize each letter πⁿ of the sequence S; whereby for synthesize of each letter πⁿ said fabrication head selectively deposits only the molecular building blocks indicated to be occurring by the binary vector representing the letter πⁿ, from said Z containers.

The system may include a container selector adapted to selectively fluidly connect to one or more of said containers to said fabrication head, to thereby enable the selective deposition by said fabrication head.

In some embodiments, building-blocks contained in the Z containers are chemically “blocked” from one end thereof so as to prevent their non-intended binding to one another. The fabrication head may be configured and operable for carrying out the following after deposition of basic molecular building-blocks of each letter at said region: washing said region to remove un-bonded molecular building-blocks deposited at the region; and applying un-blocking treatment to “un-block” the basic molecular building-blocks that are bounded to molecules at said region.

The fabrication head may be configured and operable for depositing cleavable molecules at said region prior to said synthesizing. The system may include a harvesting module configured and operable for harvesting said population of molecules from said region by cleaving said cleavable molecules.

In some embodiments, the control unit is adapted for operating said fabrication head for synthesizing, for all molecules of said population, a similar identification section. The similar identification section may include an identifying sequence of said Z types of building blocks. For example, the similar identification section may include an identifying sequence composed of molecular bases; and said plurality of containers include one or more additional containers of separately containing said molecular bases.

In some embodiments, the control unit is configured and operable for operating said fabrication head to synthesize a plurality of population of molecular sequences encoding data of a plurality of respective data blocks, at different spatially separated respective regions.

The at least one data-block may be respectively encoded by the at least one population of molecular sequences.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A to 1D are schematic illustrations and tables exemplifying the molecular data storage system according to an embodiment of the present invention;

FIG. 1E is a table showing several possible sets of short k-mer building blocks with minimal Hamming distance;

FIGS. 2A to 2C are schematic illustrations and tables exemplifying the molecular data storage system according to another embodiment of the present invention;

FIGS. 3A and 3B are schematic illustrations exemplifying according to two embodiments of the molecular data storage system of the present invention with different implementations of identification segments of the molecular sequences;

FIGS. 4A to 4C are block diagrams exemplifying the configuration of plurality of data-blocks in the data storage system according to three respective embodiments of the present invention;

FIG. 5 is a flow chare of a method for storing data according to an embodiment of the present invention;

FIG. 6 is a block diagram of a data storage fabrication system according to an embodiment of the present invention, configured and operable for fabricating a molecular data storage of the present invention and encoding data therein;

FIG. 7A is a block diagram of a data reader system according to an embodiment of the present invention, configured and operable for reading data from the molecular data storage of the present invention;

FIGS. 7B to 7D are flow charts and schematic illustrations exemplifying methods for reading data according to embodiments of the present invention;

FIG. 8 is a table showing achievable data capacities utilizing short-alphabet coding according to the present invention, with binomial alphabet encoding scheme and binary alphabet encoding schemes, as compared to standard encoding of data by molecular bases.

### DETAILED DESCRIPTION OF EMBODIMENTS

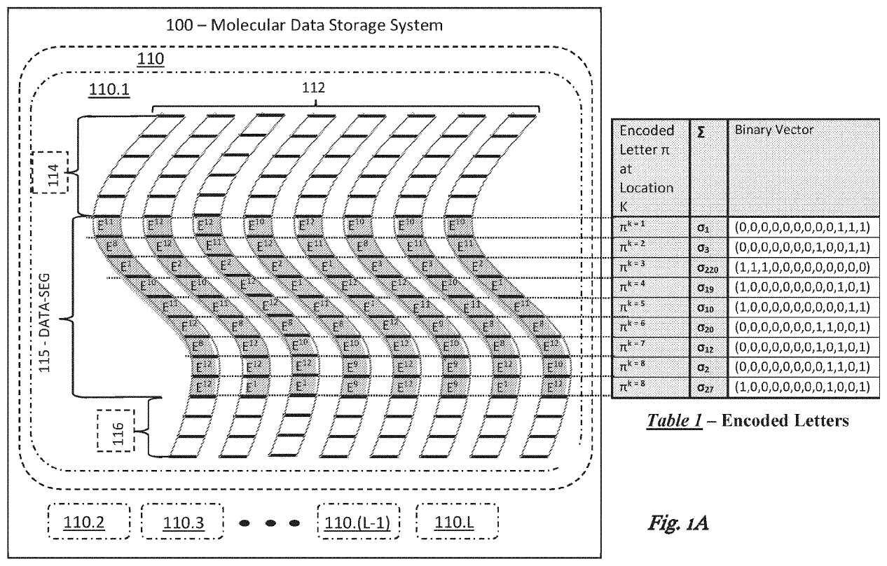

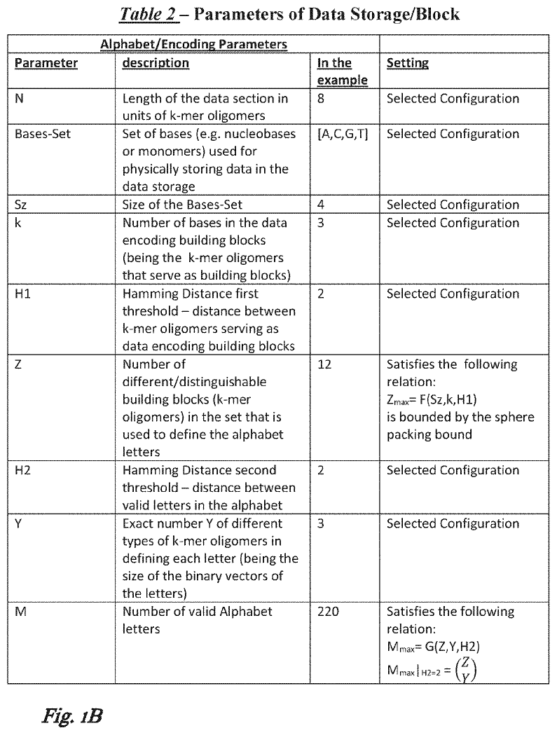

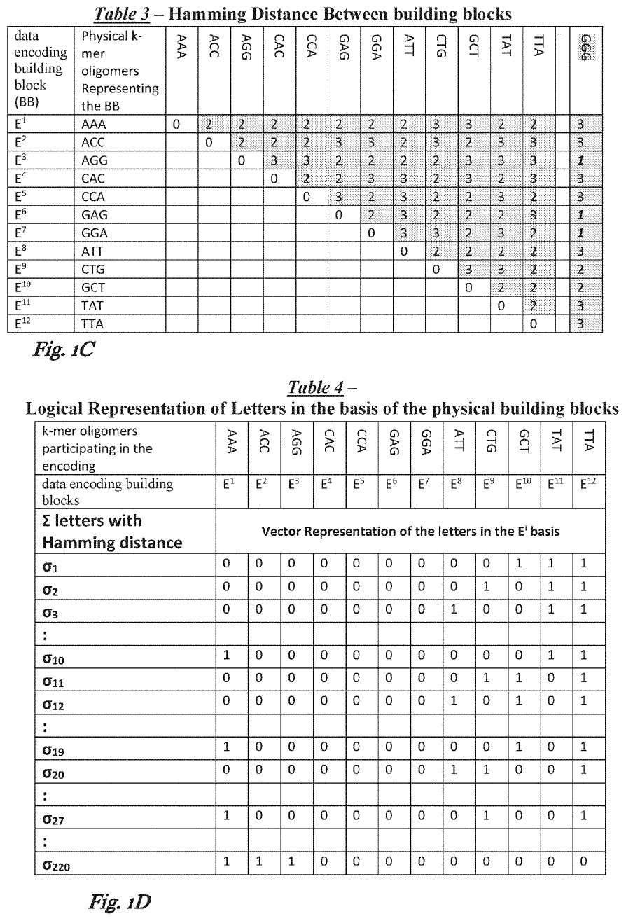

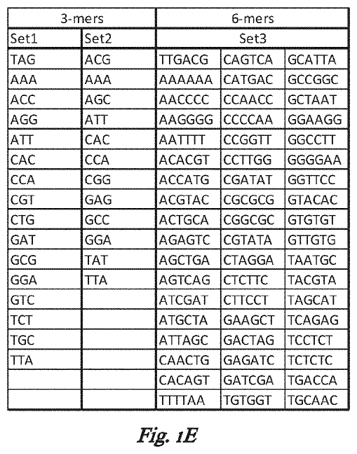

Reference is made together to FIGS. 1A to 1E illustrating data storage system 100 according to an embodiment of the present invention. FIG. 1A is a block diagram showing an example of the data storage system 100. FIG. 1B to 1D are tables showing the parameters of the data storage system 100 (FIG. 1B); the physical building blocks used to encode data in the data storage system 100 and the distances between them (FIG. 1C); And the logical representation of letters encoded in the data storage system according to occurrence of the physical building blocks (FIG. 1D).

The system 100 includes one or more data-blocks 110, whereby the term data-block is used herein to define physical element(s) encoding a block of data. Each data block, e.g. 110.1, includes a population 112 (e.g. group/collection) of molecular strands/sequences PMs by which the data of the data-block is encoded/stored. In other words, each population of molecular strands in the data storage system 100, defines a respective data-block for encoding data in the data storage system 100. In the present example there is shown data-block 110.1 with its respective population 112, and additional optional data-blocks 110.2 to 110.L (for clarity and conciseness the respective populations of molecules of the optional data-blocks 110.2 to 110.L are not specifically shown in the figure). It should be appreciated, as exemplified below, that in various embodiments of the present invention the populations of molecules of different data-blocks 110 may be located spatially separately, or the molecules of different populations may be co-located in a mixture (in the latter case, other mechanisms are provided to distinguish between molecules of different populations, this is described in more details below).

One of the data blocks, data-block 110.1 of the data storage system 100 will now be described in more detail. The data-block 110.1 includes the population 112 of physical molecular strands/sequences PMs, by which the data stored by the data block is encoded.

It should be noted that the phrases molecular strand, molecular sequence as well as polymer molecule, are used herein to indicate molecules composed of at least one chain of molecular bases (i.e. being the basic subunits of the molecule; e.g. monomers). In each molecular strand/sequence, the molecular bases are arranged in a chain/string, which generally a linear none-branched chain (although the invention may also be implemented with branched molecular strands/chains having one or more branch points at which the chains/strings are split into several strings). In any case, for clarity, each molecular strand/sequence is considered herein to include a chain/string/sequence of molecular bases.

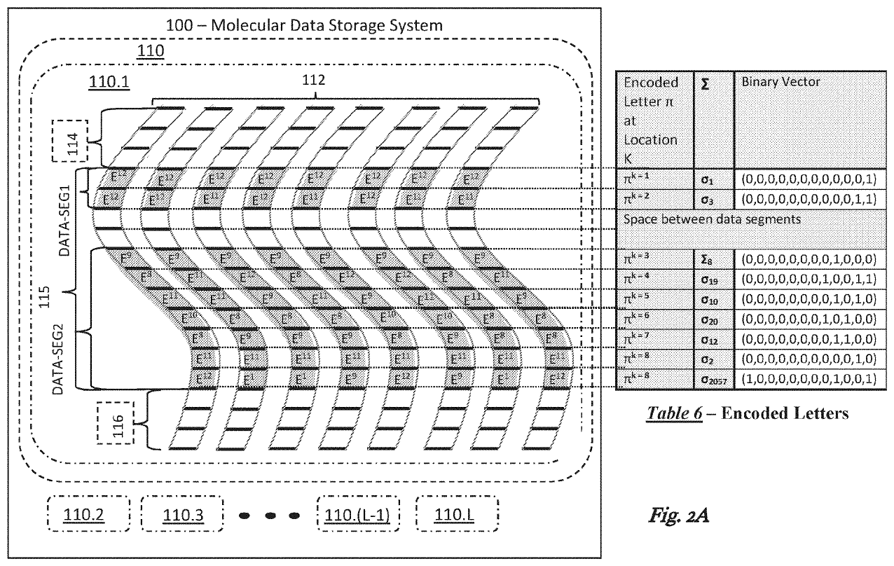

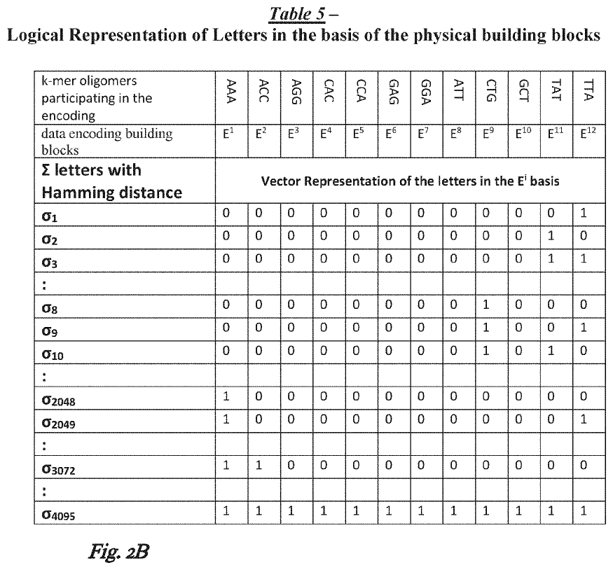

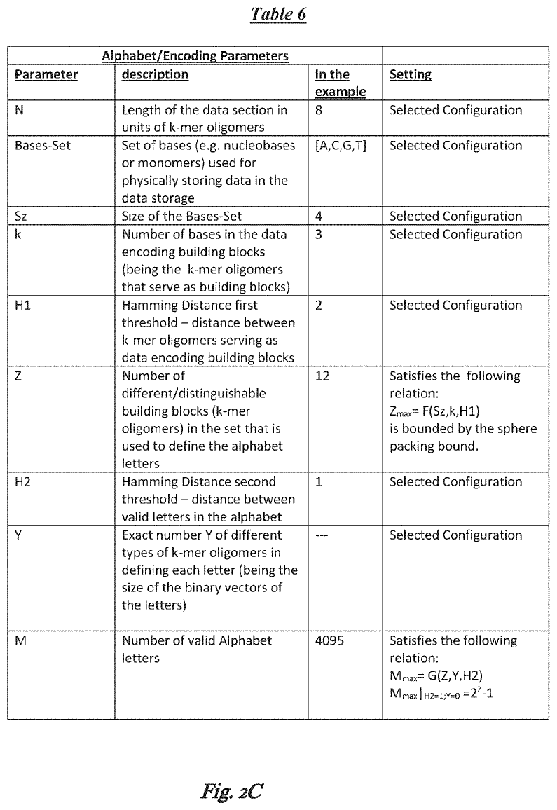

It should be understood, and as is also exemplified, that not necessarily the entire molecular strands/sequences PMs are exploited for encoding the data which is stored by the data storage. For instance, in the example of FIG. 1A only sections 115 of the molecular strands/sequences PMs are used to encode data, while other sections, of the molecular strands/sequences PMs, for instance sections 114 and 116, are non-data encoding sections. Indeed, these sections may be used for other purposes, such as population identification sections, as described below, or for RAM addressable PCR primers, or they may be non-usable sections. Also, in the example of FIG. 2A sections 115 are segmented and includes non-encoding molecular “chunks” spacing/separating between the data encoding segments DATA-SEG1 and DATA-SEG2.

To this end, the term section, used herein in relation to a part of the molecular strand/sequence, should not be considered necessarily as a continuous section of the string/chain, but may be considered to be a set of predetermined locations {n}, adjacent or not, along the molecular strand/sequence, which serve a designated purpose. For instance, the data encoding sections 115, are sections which indicate how monomer/building-blocks constituents (in different locations {n} thereof) are used to encode the data stored by the system 100. Such sections 115, as well as other sections (e.g. 114 and 116) are illustrated for clarity in the FIG. 1A as continuous, however, it should be understood that they are not necessarily continuous, but merely represent sets of predetermined locations along each of the molecular strands/sequences PMs of the population 112.

The data encoding sections 115 of the molecular strands/sequences PMs include a sequence, continuous or not continuous, of basic building-blocks {E^{z}} characterized in that all the molecular building-block in data encoding sections 115 are formed with a similar number k of plurality of molecular bases (k≥2). The different basic building-blocks {E^{z}} are distinguishable/unique basic building-blocks {E^{z}}|_{z=1 to Z} (where E^{z} is indicative of a distinguishable basic molecular building-block and z is an index running from 1 to Z for the different types participating in the data storage). Table 3 in FIG. 1C exemplifies the Z=12 building-blocks {E^{z}}|_{z=1 to 12} which are used in the present example to sort data, whereby the second column from the left in the table shows the short k-mer oligomers that corresponds/represents the building blocks.

Sections other than the data encoding sections 115 of the molecular strands/sequences PMs (e.g. sections 114 and 116) also include sequences of molecular bases however the molecular bases in those sections may not necessarily be arranged according to the arrangement of bases in the building-blocks {E^{z}}.

It is noted that in order to enable proper reading of the encoded data, the data encoding sections 115 of all the molecular strands/sequences PMs of a certain population 112 are configured with similar length N of building blocks and with predefined starting position along the molecular strands. To the similar reason, the data encoding sections 115 of all the molecular strands/sequences PMs of the certain population 112 are also configured with similar structure in terms of whether the data encoding sections 115 are continuous (i.e. having no molecular spacing between successive data blocks of the data encoding sections 115), and/or in terms of segments' lengths and the molecular spacing between the segments of the data encoding sections 115 in case the data encoding sections 115 are not continuous (i.e. distributed in segmented fashion along the molecular strands/sequences). To this end each molecular sequence of the molecular sequences of the population comprises a data encoding section (chain) 115 defining/having a continuous or non-continuous sequence of similar predetermined length of k-mers serving as the data encoding building blocks of the population 112. It is noted that in the non-limiting example of FIG. 1A the data encoding section 115 of population 112 are continuous, i.e. each includes only one segment/chain DATA-SEG. However, it should be understood that the data encoding sections 115 in the molecular strands of one or more populations 112 may not necessarily be continues. For instance, the data encoding sections 115 may be segmented and may include two or more spaced apart segments in the molecular strands separated by a non-data-encoding part of the molecular strands/sequences, such as DATA-SEG1 and DATA-SEG2 exemplified in FIG. 2B.

According to the present invention the data encoding building blocks {E^{z}}|_{z=1 to Z} which are used in the data encoding sections for encoding data, are Z different preselected k-mer oligomers (hereinafter also referred to as short-mers or short k-mers or just k-mers, interchangeably) of similar predetermined sizes/lengths k of molecular bases. According to the present invention a plurality k≥2 of bases are included in each short k-mer that serves as a data encoding building block E^{z}. In the present example k=3; i.e. k=3 bases are included in each distinguishable short k-mer that serves as a data encoding building block E^{z}. In Table 3 of FIG. 1C, the second column form the left exemplifies different arrangement (bases constituents and/or order) of Z=12 building-blocks {E^{z}}|_{z=1 to 12} each formed with k=3 molecular bases. The molecular bases in this particular non limiting example are all belonging to the base-set [A,C,G,T] of nucleotides (also known as and referred to herein and Cytosine, Guanine, and Thymine nucleotides). Here exemplified is the use of a base-set [A,C,G,T] with size Sz=4 (number of bases (elements/monomers)).

The table in FIG. 1E further exemplifies other possible building blocks sets of short k-mer with minimal Hamming distance H1>1 between the short-mer building blocks. Set 1 and Set 2 in the table are two building block sets of 3-mers (k=3) with minimal Hamming distance of 2. The sizes of the sets are 16 and 12 for Set 1 and Set 2 respectively. Set 3 on the right-hand side of the table is a building block set of 54 6-mers (k=6) with minimal Hamming distance of 4.

It should be understood that the types of molecular base-set (i.e. the types of bases) used in the building-blocks {E^{z}}|_{z=1 to 12} of data storage system 100, may be different from implementation to implementation of the system depending on various prerequisites required from the data storage system. For instance, the molecular strands PMs, or the data encoding sections 115 thereof, may be bio-polymers, such as nucleic acid, DNA or RNA, which are poly-nucleotide molecules constructed with set of bases (hereinafter base-set) including Adenine, Cytosine, Guanine, and Thymine nucleotides (A,C,G,T) (e.g. as in DNA), or with a base-set including Adenine, Cytosine, Guanine, and Uracil nucleotides (A,C,G,U) (as in RNA). The sizes Sz of each of these base-set is Sz=4. In other embodiments, the molecular strands/sequences PMs, or the data encoding sections 115 thereof, may include different base-set with different size Sz and/or with different/other types of polymers/monomers, bases e.g. bio-polymers/monomers or synthetic-polymers/monomers. The Base-Set may include any number Sz>1 of molecular bases (e.g. the molecular bases in the base-set may be bio-monomers or synthetic monomers with any number as may be permitted by the chemistry of the specific set of bases that is used). To this end, the data encoding sections 115 may be implemented according to the present invention using base-set including or consisting of the A, C, G, and T nucleotides, and/or the A, C, G, and U nucleotides, and/or with these nucleotides plus additional one or more bases, or with different sets of molecular bases, being e.g. bio-type monomers and/or other monomers, e.g. synthetic⁶⁻⁸. The data of the data-block 110.1 is encoded by the sequences/chains of basic molecular building-blocks in the molecular strands/sequences PMs of the data-block population 110.1. According to the present invention the data encoding sections 115 of the molecular sequences of the population, encode collectively (together) a sequence of encoded alphabet letters S=(π¹, π², . . . , πⁿ . . . , π^{N-1}, π^{N}). To this end, it would be appreciated that the data encoding building block E^{z} (formed by the short k-mer of predetermined k of bases of the base set) are the physical building blocks by which data is encoded. However, each single data encoding section 115 of any one molecular sequence does not by itself encode data. Instead, the data is encoded collectively by the plurality of data encoding sections 115 in the population 112. In other words the encoded letters S=(π¹, π², . . . , πⁿ . . . , π^{N-1}, π^{N}) are logically collectively encoded in to the population 112, and not by any single data encoding section.

In some implementations the data of the data-block 110.1 is encoded in an ordered sequence S=(π¹, π², . . . , πⁿ . . . , π^{N-1}, π^{N}) of letters {πⁿ} encoded in the population 112 of molecular strands/sequences PMs. The encoded letters {πⁿ} are generally associated with, or belong to, an alphabet Σ that is used for encoding the data. An example of a definition of such alphabet Σ is exemplified in Table 4 of FIG. 1D, and will be discussed in more details below.

As indicated above each of the data encoding sections 115 of the population 112 includes a sequence (continuous or not) of a similar length N of building blocks. The plurality of data encoding sections 115 of the population 112 (not any single one of them alone but not necessarily all of them) encode together the data sequence S=(π¹, π², . . . , πⁿ . . . , π^{N-1}, π^{N}) of encoded letters {πⁿ}. Considering that the positions of the building blocks along the data encoding sections 115 of the population 112 are indexed by n=1 to N, an encoded an letter at position n along the encoded data sequence S can be determined by comparing the building blocks occurring/existing at the position indexed n in the plurality of data encoding sections 115 of the population 112 to the definition of the alphabet letters Σ used of encoding the data.

In the present invention, each valid encoded alphabet letter πⁿ at location n of the sequence S of alphabet letters is characterized by occurrence of a certain different types of short-mers of the building-block-set in the corresponding location n along the data encoding sections of the plurality of molecular sequences of the population. Thus, the encoded letters {πⁿ} are encoded by the order of the Z types of basic molecular building-blocks {Eⁿ}|_{n=1 to Z} arranged at least in parts of the plurality of molecular strings/strands/sequences PMs of the population 112. Nonetheless, the population includes non-similar molecular sequences PMs (which as said above together encode the data of the population). Therefore, according to the technique of the present invention, the size M=|Σ| of the alphabet Σ (namely number of distinct letters therein) is greater than the number Z of different types of basic molecular building-blocks that are used/included in the molecular strands/sequences PMs, (M>Z).

This is achieved by exploiting the redundancy of molecular strands/sequences PMs in the population 112, to define the letters in the alphabet Σ in terms indicating occurrence of each of the Z types of basic molecular building-blocks in the letter. In this manner, the number of M of different letters which are defined in the alphabet Σ may be higher than the number Z of basic molecular building-block types.

In other words, according to the present invention, the alphabet Σ letters {σₘ}|_{m=1 to M} can be represented/defined by respective distinct subsets over the space spanned by the Z different types of short-mer building blocks {E^{z}}|_{z=1 to Z}. Accordingly, each valid letter πⁿ encoded at location n of the sequence S is characterized by occurrence (in the location n along the data encoding sections of the molecular sequences of said population) of a plurality of different types of short-mer building blocks {E^{z}} of the building-block-set, which matches the binary vector representation of one of the predefined alphabet τletters {σₘ}|_{m=1 to M}.

This is exemplified in self-explanatory manner in FIGS. 1A and 1D, whereby FIG. 1A the data encoding sections 115 of data block 110.1 are depicted to exemplify sequences of building blocks {E^{z}} in population 112 of molecular strands in this data block 110.1; The data encoding sections in this none-limiting example are of length N=8 building blocks, and Table 1 in FIG. 1A shows the letters {πⁿ}|_{n=1 to N} encoded by each position n of the data encoding sections 115, with the binary vector representation of the building blocks occurrence. Table 1 also shows the matching of this occurrence vector with the letters of the predefined alphabet Σ according to the definition of the alphabet Σ={σₘ}|_{m=1 to M} in provided in Table 4 of FIG. 1D.

Conventional molecular storage techniques (e.g. such as disclosed in^{1-3,9}), encode the data using an alphabet whose size is equal to or smaller than the number of bases of the molecular sequences. In other words, in such conventional techniques there is one-to-one correspondence between the alphabet letters and the bases.

According to the present invention, the number M of letters in the alphabet Σ is greater than the number Sz of bases in the base set and is also greater than the number Z of the basic molecular building blocks {E^{z}}. More over there is no one-to-one correspondence between letters and bases and no one-to-one correspondence between letters and the basic molecular building blocks. This is achieved by exploiting the redundancy of molecular strands/sequences PMs in the population 112, to define the letters in the alphabet Σ in terms of the occurrence generally more than one of the Z types of basic molecular building-blocks in each the letter. In this manner, redundancy of the data encoding in the molecular strands/sequences PMs of the population 112 is reduced, but the number of M of different alphabet Σ letters which are used for the encoding is increased (i.e. it may be much higher than the number Z of different basic molecular building-block and much higher than the number of bases), thus yielding increased/improved data density on the expense of somewhat reduced redundancy. Indeed, as the redundancy of data encoding in molecular storage systems are generally inherently higher than necessary for error correction, the reduced redundancy provided by the technique of the present invention has no negative implications, and on the other hand, the improved data density provide a significant advantage.

Turning now to FIG. 1D, there is provided a table, Table 4, exemplifying a definition of an alphabet Σ according to an embodiment of the present invention. In the present example alphabet Σ is constructed/defined based on Z=12 different basic building-block: {E^{z}}_{z=1 to Z=12}, which are different short k-mers, each having an order structure of k=3 bases selected from the base set {A, C, G, T} (e.g. where A, C, G, and T stand for the Adenine, Cytosine, Guanine, and Thymine monomers of the DNA). It would be appreciated by those skilled in the art, that generally without departing from the scope of the present invention, a different set of parameters may be used in various implementations of the invention, such as a different set of bases with the same or different number of bases; a different number of k bases in each building block; and/or different building-blocks set {E^{z}} or a different number of selected Z thereof may be used in the definition of letters.

Each row in Table 4 represents the definition of a different letter σₘ in the alphabet Σ in terms of a binary occurrence vector of the basic molecular building blocks {E^{z}} participating in the physical encoding of this letter. Each column in Table 4 presents a different one of the building blocks {E^{z}} with its corresponding physical k-mer representation, and each letter defining row indicates which of the building blocks occur in the respective letter (marked 1), and which does not occur (marked 0), thus yielding the binary occurrence vector definition of the letter.

In the example of Table 4 there are defined M=220 letters, which are defined according to the following parameters of the alphabet presented in Table 2 of FIG. 1B: Sz=4; k=3; H1=2; Z=12; H2=2; Y=3. It would be appreciated that these parameters are configuration parameters which may be different from implementation to implementation of the present invention (although some bounding relations between those parameters may exist as described in more detail below).

In a preferred embodiment of the present invention, the alphabet letters Σ={σₘ} used to encode data in the data storage are predefined according to what is referred to herein interchangeably as a binomial encoding scheme or exact Y parameter. In the binomial encoding scheme the alphabet is defined with an exact number parameter Y such that each alphabet letter σₘ includes, or is identified by, a predetermined exact and constant number Y of a plurality of different building blocks of the building-block-set {E^{z}} (i.e. presented by Y different types of short-mers whereby Y≥2) in all the letters {σₘ}_{1 to M}. Accordingly, each valid encoded alphabet letter πⁿ at location n of the sequence S of encoded alphabet letters is characterized by occurrence of the exactly Y of a plurality of Z different building blocks at the corresponding n^{th} locations of the data storing sections/segments 115 of the molecular sequences/strands/strings PMs (e.g. monomer strings) in the population 112.

Referring to FIG. 1A, the data storing sections/segments 115 of the molecular sequences/strands PMs are shown together with indications to the types of the building blocks from the building-block-set {E^{z}} which are arranged in the data storing sections/segments 115. FIG. 1A presents a preferred embodiment of the present invention in which all the alphabet letters Σ={σₘ} are defined to have a similar exact number Y of a plurality different building blocks {E^{z}} in each letter σₘ. In this non-limiting example the exact number Y of different building blocks {E^{z}} in each letter σₘ is set to be Y=3 although it would be appreciated that other selection of the parameter Y is possible without departing from the scope of the present invention. Thus, Table 1 in FIG. 1A illustrated the sequence S of the encoded letters πⁿ in data storing sections/segments 115 of FIG. 1A, and their respective binary vectors' representation. As would be appreciated, the weight of those binary vectors are all the same and match the exact number Y (which is 3 in this example). To this end in some embodiments of the invention the set of predefined alphabet letters Σ≡{σₘ}|_{m=1 to M} consists only of binary vectors (occurrence vectors) of equal weight.

Indeed, it is generally not necessary to define the alphabet letters Σ={σₘ} with the exact number parameter Y in such a way that all of them having a similar exact number Y of building blocks {E^{z}}. FIGS. 2A to 2C show an embodiment where the alphabet letters E are defined without imposing a similar number Y of building blocks in each. Such alphabet definition, which do not implement the exact number parameter Y is referred herein as a binary encoding scheme. The term binary encoding scheme should not be confused with the phrase binary vector or binary vector components, as these letter terms pertain only to a manner of representation of the alphabet letters as binary vectors, and such representation may be implemented in both the binomial and the binary encoding schemes.

Thus the present invention may be implemented utilizing the following alphabet encoding schemes:

I. Binary Encoding Alphabet (e.g. as Exemplified in FIGS. 2A to 2C)

The synthesis process allows for each of the Z short k-mers in the building block set {E_{z}}_{z=1 to Z} to be either included or not in every position n of the synthesized molecular sequences of the population (i.e. to be either included or not in every synthesis cycle). This yields an effective output alphabet of size M=|Σ|=2^{Z}−1 letters (in this case each letter in an encoded data sequence encodes Z−1=└log₂(|Σ|)┘ bits). Accordingly, encoding an r-bit binary message utilizing this alphabet requires

$$\frac{r}{{log}_{2} ( \vert \sum \vert )} = \frac{r}{{log}_{2} ( {2}^{Z} - 1 )} \approx \frac{r}{Z}$$

synthesis cycles (utilizing the short-mers building blocks {E_{z}} as the basic elements of the synthesis). To this end, in terms of the molecular bases (e.g. [A,C,G,T], the length of the data encoding sections 115 of the molecules of the population encoding such massage would accordingly be:

$$length = k * \frac{r}{{log}_{2} ( {2}^{Z} - 1 )} ,$$

where k is the number of bases in the k-mer building blocks (this is while ignoring error correction/redundancy codes such as Reed-Solomon code which may be introduced into the complete encoded/stored/sent message). In other words, every b=Z−1=└log₂(|Σ|)┘ bits will be encoded as a single letter in the output/encoded sequence S of letters.

II. Binomial Encoding Alphabet (e.g. as Exemplified in FIGS. 1A to 1E)

The synthesis process requires that exactly Y distinct short k-mers to be included in every position n of the synthesized molecular sequences of the population (i.e. exactly Y distinct short k-mers are included in an synthesis cycle). Therefore, every letter in the alphabet is a subset of size Y of the short k-mer building blocks {E_{z}}_{z=1 to Z}. This yields an effective output alphabet of size

$$M = \vert \sum \vert = ( \begin{matrix}Z \\ Y\end{matrix} )$$

letters. Encoding an r-bit binary message utilizing this alphabet requires

$$\frac{r}{{log}_{2} ( \vert \sum \vert )} = \frac{r}{{log}_{2} ( ( \begin{matrix}Z \\ Y\end{matrix} ) )}$$

synthesis cycles (utilizing the short-mers building blocks {E_{z}} as the basic elements of the synthesis). To this end, in terms of the molecular bases (e.g. [A,C,G,T]), the length of the data encoding sections 115 of the molecules of a population encoding such massage would accordingly be:

$$length = k * \frac{r}{{log}_{2} ( ( \begin{matrix}Z \\ Y\end{matrix} ) )} ,$$

where k is the number of bases in the k-mer building blocks: (this is without considering error correction/redundancy codes such as Reed-Solomon code which might be included in the message). Intuitively, every

$$b = ⌊ {log}_{2} ( ( \begin{matrix}Z \\ Y\end{matrix} ) ) ⌋$$

bits will be encoded as a single letter in the output/encoded sequence of letters.

It should be noted, as would be evident to one skilled in the field, that in practice for both encoding schemes the data capacity may be extended by encoding the binary message, of length r, in larger blocks.

Thus, utilizing the Binary encoding scheme/alphabet, permits to define much greater number of letters as compared to the Binomial encoding scheme/alphabet, and thus enable to increase the data density of the data storage (on expanse of further reduced data encoding redundancy). For instance, in the example of FIG. 2A where the exact number Y parameter is not imposed, the number M of letters which can be defined is 2^{z}−1 where Z is the number of building blocks (Z=12 in the both examples of FIGS. 1A and 2A) would yield M=4095 different letters (see E.g. Table 5 in FIG. 2B). In turn this would permit a much higher data density (without imposing the exact Y=3 parameter exemplified in the embodiment of FIG. 1A, the achieved data density in the embodiment of FIG. 2A may be about log(M_{Fig.2A}=4095)/log(M_{Fig.1A}=220)˜1.54 times higher as compared to the embodiment of FIG. 1A).

Nonetheless, in preferred embodiments of the present invention the alphabet letters Σ={πₘ} are defined with the Binomial encoding scheme, i.e. with a constant exact number Y of building blocks {E^{z}} in each letter, whereby Y≥2. In this regards, the inventors' of the present invention have realized that using alphabet letters Σ={σₘ} defined in this way (with an exact similar number of plurality of Y different building blocks in each letter) facilitates the use of a more robust and efficient sequencing protocol when reading the data stored in the data storage.

In this regards the improved efficiency resulting for the exact numerical parameter Y is provided at least in terms of the sequencing protocol, which may be employed in this case for reading the encoded data. Indeed in this case the reading might be conducted by carrying out sequencing the molecular strands of each population only as until exactly Y′≡Y of different building blocks are actually found/encountered per each location n in the data encoding sections 115 of sequenced molecular strands of the population, and can be confidently stopped once the actual number Y′ₙ of different building blocks encountered in each position n exactly matches the exact number parameter Y (this will yield 100 percent confidence that all the encoded letters are properly read). For proper reading, sequencing will be continued at least until the actual number Y′ of different building blocks encountered in at least one position n is not smaller than the parameter Y. To this end the condition for enabling sequencing stop is for any position n Y′ₙ≥Y (Y′ₙ≡Y presents a properly read letter at location n and Y′ₙ>Y presents an invalid letter read from location n which might optionally be corrected using various validation techniques). On the contrary, in embodiments such as of FIG. 2A where the binary encoding scheme is employed (where no exact number parameter Y is imposed), very large numbers of molecules of the population would need to be sequenced in order to achieve high confidence or otherwise only a partial confidence level may be statistically assumed (typically 70% to 90%). In this regards, see the description below regarding the sequencing depth R which may be used in each of the encoding schemes.

Moreover, this technique also improves the robustness of the data reading in terms of the ability to validate with confidence that the read letters are correct and no-miss reading or miss writing occurred. This is because until the sequencing process of the reading is stopped, generally most of the building blocks of each encoded letters have being already redundantly sequenced several times (except may be the last building block of the last read letter which was sequenced and by which permitted the stopping of the sequence protocol of the reading). Accordingly, in cases where during the reading/sequencing it is found that in at least one position n, the actual number Y′ₙ of different short-mers (the building blocks) is not equal to Y (i.e. in case where for at least one n Y′ₙ≠Y of different types of short-mers is encountered in at least one position n along the plurality of molecular strands), in that case it can be assumed that the alphabet letter 70 read from this position is invalid. For the specific case where Y′ₙ<Y for any one or more n(s), the stopping condition of the reading/sequencing is met and thus in some implementation the sequencing would be continued until all of the molecular strands in the population are sequenced or until the condition Y′ₙ=Y is fulfilled. If after all of the molecular strands in the population are sequenced still for some n, Y′ₙ<Y, in that case encoded letter read from the location n can be determined to be invalid/erroneous. Also in the specific case where Y′ₙ>Y for any one or more n(s), the encoded letters read from the location n(s) for which Y′ₙ>Y may be assumed invalid. However in that case it may still be possible to apply a certain statistical error correction procedure to assess/estimate with more or less confidence what are the actual correct letters encoded in these locations (e.g. based on the number of times each basic building block is encountered at the corresponding location. To exemplify such error correction, one may consider for instance the case where at the position n=1 the following basic building blocks are encountered the following number of times during sequencing of the population 112:

E¹⁰—encountered 217 times;

E¹¹—encountered 121 times;

E¹²—encountered 150 times; and

E⁸—encountered 3 times;

In that case the number of times E⁸ is encountered is clearly relatively minor, and is an order of magnitude or more less than the number of times any other of the building blocks where encountered. Accordingly, the sequencing or synthesis of E⁸ at that location n=1 may be assumed to be erroneous, and be thus neglected. In this case the encoded letter at location n=1, πⁿ⁼¹, might be correctly determined to be σ₁ as shown in Table 1. Other error correction procedures for correcting the reading of invalid letters of which Y′ₙ>Y might be carrying our resequencing of a different part of the population 112, or otherwise setting an predetermined statistical threshold based on the absolute or relative number of times the building blocks are encountered, and determine based on this threshold which of the read building blocks is significant (e.g. E¹⁰, E¹¹, and E¹² in the above example), and which are not and can be ignored (e.g. E⁸ above).

As indicated above, in the molecular data storage system of the present invention the basic molecular building blocks {E^{z}}_{1 to Z} are Z different preselected k-mers (short-mers) of similar predetermined sizes/lengths k of molecular bases, with a similar plurality of preselected k≥2 bases in each building block/k-mer. One important advantage that this feature of the present invention provides, is that there are more data encoding building blocks, than possible with other techniques in which the molecular bases (e.g. the [A,C,G,T] bases) themselves serve as data encoding building blocks (in the present example there are Z=12 building blocks and only Sz=4 bases). This in turn facilitates higher speed/rate of data writing/encoding in to the data storage system (this is because the synthesis of the molecular population 112, or at least the synthesis of the data encoding sections of the population may be carried out with the short-mers serving as the building blocks instead of the bases themselves—and therefore longer molecular chains/strands may be synthesized with improved speed). Another important advantage is that by this technique the Z different types of short-mers selected as to participate as data encoding building blocks of the usable building-block-set {E^{z}}_{1 to Z} may not necessarily include all the possible short-mers (k-mers) having the predetermined length k which may be constructed out of the molecular bases, but the usable building-block-set {E^{z}}_{1 to Z} may be selected such that it includes a sub-set of all the short-mers of the predetermined length k, satisfying that a hamming distance between each short k-mer in the subset, which is selected as the building-block-set, and any other short k-mer in the building-block-set (i.e. in the selected subset) is greater or equal to a certain first H1 threshold of minimal hamming distance. Utilizing the building-block-set consisting only of building blocks with minimal hamming distance H1≥1 between them facilitates robust reading of the encoded data with improved error correction. To this end, in both the examples of FIGS. 1A and 2A the building-block-set consists only of building blocks {E^{z}}_{1 to Z} with minimal hamming distance H1≥2 between them.

Table 3 in FIG. 1C shows the building block set {E^{z}}_{1 to 12} that are used to encode data in the polymeric data storage systems of FIGS. 1A and 2A. In these none-limiting examples, the building blocks {E^{z}} are oligomers with k=3 bases selected from the base-set [A,C,G,T] such that the minimal hamming distance threshold H1≥2 between each two different building blocks E^{z1} and E^{z2} (z1≠z2) in the building block set {E^{z}}. The first column in the table shows the building block and the second column the corresponding short k-mer serving as that building block. The first/title line also shows k=3 short-mers of the base set [A,C,G,T], and the bulk of the table presents the hamming distances between the short-mer building in the second column and those of the first row. As shown all the short k-mers serving as building blocks have hamming distance H1≥2 between them. The short k-mer ‘GGG’ does not serve as a building block and is not included in the building block set (e.g. does not appear in the second column of the table) since its hamming distance from at least one other building block (e.g. E3, E6 and E7) is smaller than the minimal hamming distance threshold H1=2.

Thus, in some embodiments the building-block-set {E^{z}}_{1 to Z} is selected to include only short-mers of similar length k, satisfying that a hamming distance between each short k-mer E^{z1} in the building-block-set {E^{z}} and any other short k-mer E^{z2} in the building-block-set {E^{z}} that is greater or equal to first of minimal hamming threshold H1=2. In some embodiments, typically with k larger than 3, the minimal hamming threshold may be set to H1=3 or to H1=4 or even higher, so as to provide improved data validation/correction. It is noted that the minimal hamming threshold H1 between the building-blocks should be generally smaller or equal to the length k of the short k-mers H1≤k, and that preferably in typical embodiments of the present invention the minimal hamming threshold H1 between the building-blocks is set to be strictly smaller than k, H1<k, so that a sufficient plurality of data encoding building-blocks is included in the building-block-set {E^{z}} to enable high enough data density.

Further to the above indicated condition/configuration that the different types of preselected short-mers in the building-block-set has similar plurality of preselected k≥2 bases, in some preferred embodiments of the present invention similar predetermined size k of the short-mers in the building-block-set is also selected such that k does not exceeds 20 bases, namely k≤20, and more preferably k≤10. This is important in order to enable production scale data storage via molecular synthesis as well as low physical density. Indeed in order to properly exploit data encoding with higher k values, one would be required to use all or most of the large plurality basic building block short-mers with these higher k which satisfies the required hamming condition. However with large k, e.g. larger than 20, the number of such building blocks would be very large requiring cumbersome synthesis machines for carrying out the data encoding process. For instance if building blocks short-mer of k=10 are defined over a base set of size Sz=4 with the minimal hamming distance H1=3 between them, this would result in a large number of Z≥10,000 of building blocks in the data encoding building-block-set {E^{z}}1 to Z. This in turn would require a cumbersome synthesizing machine. Thus, in preferred embodiments of the invention size k of the short-mers in the building-block-set is limited to be k≤7, and more preferably k≤5. In the none-limiting embodiments of FIGS. 1A and 2A, k=3 is selected. This specific oligomer size k=3, has a particular advantage specifically in cases where biochemical bases such as [A,C,G,T] or [A,C,G,U] are used because trimers of such biochemical bases are readily available for commercial use.

As indicated above in some embodiments, as illustrated in the example of FIG. 1A, the set of predefined alphabet letters Σ≡{σₘ}|_{m=1 to M} used to encode data in the molecular data storage system is selected such that it consists only of binary vectors of similar weight, and/or such that all the alphabet letters {σₘ} are defined with a similar exact number Y of different building blocks. In this case essentially the hamming distance between the letters is at least H2=2.

Alternatively, or additionally, in some embodiments, as illustrated in the example of FIG. 2A the exact number parameter Y is not enforced in the definition of the letters. None the less in some cases a certain minimal hamming distance between letters may be enforced in order to avoid ambiguity between letters. Thus in some embodiments the predefined alphabet Σ of letters {σₘ}|_{m=1 to M} used to encode data in the molecular data storage system are selected so that it consists only of binary vectors having hamming distances between them greater or equal to a certain second threshold H2, whereby the second threshold H2 of minimal hamming distance is at least (H2≥2). In some embodiments the second threshold H2 of minimal hamming distance is at least (H2≥3). In FIG. 2A the second threshold H2 of minimal hamming distance is H2=1.

Table 2 in FIG. 1B summarizes in self-explanatory fashion the parameters discussed above of the data storage system of the embodiment of FIG. 1A. It is noted that for clarity the parameter N, which represents the length of the data encoding section in units of k-mer short-mers (trimers in this case) is relatively short N=8, in order to enable depiction of the data sections in the figure. It is understood that in practical implementations this parameter would generally be one or several orders of magnitude larger to allow storage of substantial amount of data by the population 112 of each data block, e.g. 110.1, of the data blocks 110 of the data storage system.

Referring now more specifically to FIGS. 2A to 2C, the embodiment of the present invention shown in these figures exemplifies a more general implementation of the invention, which is also based on the short-mers serving as the data encoding building blocks {E^{z}}_{1 to Z} (e.g. with k preferably not exceeding 20 and more preferably not exceeding 10. In this embodiment and related figures, the similar notation is used to designate the similar elements/modules as described above with relation to FIG. 1A, and thus the above description is to be considered relevant to this embodiment as well, except for the differences discussed below. Moreover, as can be seen by comparing the Tables 2 and 6 (in FIGS. 1B and 2C) the following configuration parameters of the basic building blocks are selected similarly in both of the embodiments of FIGS. 1A and 2A:

Sz=4 (being the size of the base-set); k=3 (being the number of bases in each data encoding building blocks {E^{z}}); H1=2 (being the minimal hamming distance threshold between building blocks). Accordingly, the similar number of data encoding building blocks {E^{z}}_{1 to Z} is defined. For clarity, in this none-limiting example also the same base-set [A,C,G,T] and the same combinations of bases in the building blocks {E^{z}} are used as in the embodiment of FIG. 1A, and thus Table 3 of FIG. 1C illustrating the Z=12 short k-mer building blocks is also relevant to the embodiment of FIG. 2A.

The main difference between this embodiment of FIG. 2A and the embodiment of FIG. 1A is that in the present embodiment of FIG. 2A the parameters of the letter definition over the building blocks space are different. More specifically, the exact-number parameter Y of the letter's definition as well as the second minimal hamming distance H2 between the letters are not required/enforced in this embodiment. As indicated above, on the one hand the Y and/or the H2 parameters of the alphabet, facilitate data validation and/or error correction of the read/sequenced data and moreover the Y parameter facilitates employment of fast and definite reading protocol when sequencing the molecular population, while on the other hand imposing these parameters on the alphabet reduces the number of the letters thus reducing the data density.

Indeed, in some embodiments of the present invention the exact number parameter Y may not be imposed, in order to achieve greater number of letters and accordingly higher data densities. In such embodiments the data validation and error correction may be implemented achieved for instance by using a second minimal hamming distance threshold H2≥1 greater than 1 between the letters, and/or by introducing data redundancy to the encoded data and/or introducing error correction codes to the encoded data (according to any known in the art error-correction/data-validation technique).

However, the Inventors of the present invention consider that in some implementation the embodiment of FIG. 1A may be preferred, since implementing the alphabet letters with the exact number Y of different building blocks is not only advantageous in terms of the data-validation/error-correction abilities, but has also significant advantages in terms of the speed and efficiency of reading/sequencing of data stored in a molecular data storage configured with the exact number parameter Y.

Another feature exemplified in FIG. 2A, relates to that in this embodiment the data encoding sections 115 of the molecular strands/sequences of the population is explicitly shown to be segmented to several segments DATA-SEG1 and DATA-SEG2. It should be understood that this feature is a matter of configuration, and may also be implemented as such in the data encoding sections 115 of the embodiment of FIG. 1A, or alternatively the embodiment of FIG. 2A may be implemented with the continuous, none-segmented data encoding sections 115.

Referring together to all the embodiments of the data storage system 100 of the present invention, it should be noted that in some implementations such data storage systems 100 may be configured and operable for storing large amounts of data and may include a large number of data blocks (populations). Alternatively or additionally, in some implementations the data storage systems 100 may be configured and operable for use as a molecular mark/label or tag (e.g. marker/tag) which can be applied on or within an object which is to be marked/labeled, and/or optionally embedded within the material constituting the object, for labeling the object and for enabling its identification or verification. In this case the data storage system 100 may include at least one data-block (e.g. as few as one population of molecular sequences), by which the marking data indicative of the molecular mark is encoded. In some embodiments the molecular tag or label further includes, in addition to the data storage system 100, also additional constituent materials selected/designed for embedding and/or binding the molecular mark on an object in a designated way. The additional constituent materials may include for instance material that encapsulates the coding material and protects it against degradation as is described in U.S. Pat. No. 9,850,531. It should be emphasized that this invention provides for using composite encoding within such tagging systems, enabling more tagging flexibility.

As also shown in FIGS. 1A and 2A, the data storage system 100 may include a plurality of populations of the molecular strands/sequences defining a respective plurality 110 of data-blocks encoding data in the data storage system 100. For example, in each data-block/population there may be typically (e.g. using current controlled polymer synthesis technologies) in the order of 10⁵ to 10⁸ molecular strands/sequences PMs. The usable length for storing data in the molecular strands/sequences (i.e. the lengths of the data encoding sections 115) may be in the order/range of about N=20 to 1000 building-blocks, when considering the present techniques and technologies for controlled polymer synthesis (note that in the embodiments discussed above only short data encoding sections 115 are presented for clarity with lengths N=8). Accordingly, considering alphabet of size M, data capacity of about

$$D C = ⌊ \frac{N}{{log}_{M} ( 2 )} ⌋ bits = ⌊ \frac{N / 8}{{log}_{M} ( 2 )} ⌋ bytes ,$$

where N is the lengths of the data encoding sections 115 of the population (e.g. with 20 to 1000 building-blocks as said above) can be stored by each such population. Typically, in most cases, a plurality of such populations/data-blocks 110 are included in the data storage system 100 to facilitate storage of large amounts of data.

Thus, in the embodiment of FIGS. 1A and 2A with lengths of only N=8 of the data encoding sections 115 of the population 112, data capacity of about DC_{Fig.1A}=62 bits, DC_{Fig.2A}=95 bits. The data density per building block of about

$${DD}_{BB} = \frac{1}{{log}_{M} ( 2 )} bits$$

which is about 7.75 bits per building block in FIG. 1A and about 11.87 bits per building block in FIG. 2B. For comparison with techniques which do not utilize the short k-mers of k>1 as building blocks, the data density per base is given by

$${DD}_{Base} = \frac{1}{k * {log}_{M} ( 2 )} bits ,$$

which is about 2.58 bits per molecular base in FIG. 1A and about 3.95 bits per base in FIG. 2A. As said above the embodiment of FIG. 1A has indeed somewhat lower data density by facilitates use of improved reading/sequencing protocol as well as improved data correction/validation.

As indicated above, indeed, in some implementations, the data storage is configured to store large amounts of data and include a plurality of building blocks 110 with different respective populations {112} of molecules storing data.

In some embodiments the different respective populations {112}, which are associated with different data-blocks 110, reside at different physical regions/places, and can thus be distinguishable based on their location/region. For instance, the populations may be stored in separate regions of a matrix/plate carrier or on different containers, such that molecules of different populations {112} can be separately read/sequenced from the different locations.

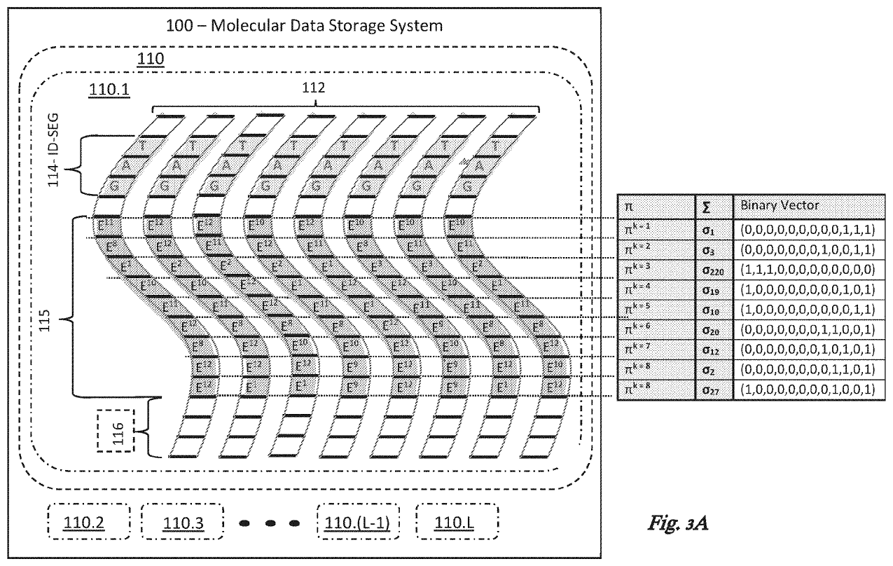

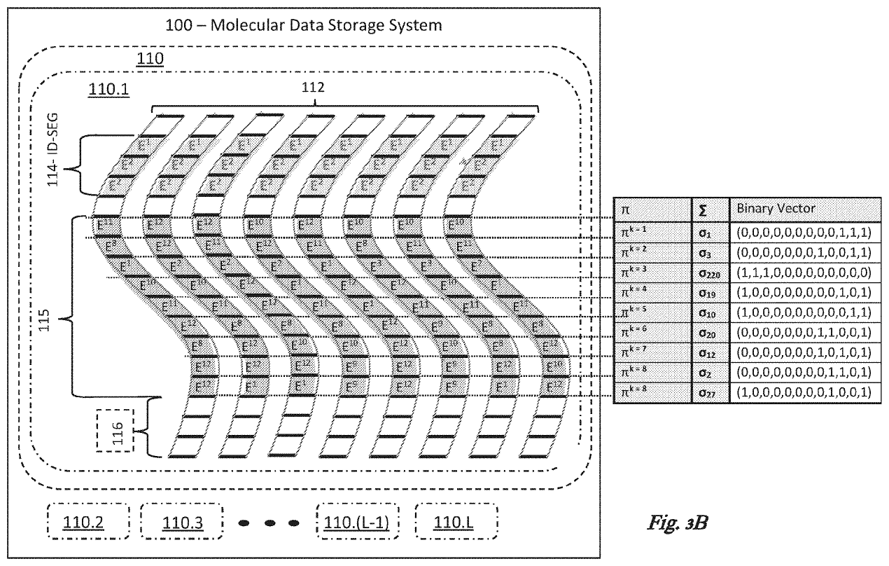

Alternatively, or additionally, as shown in FIGS. 3A and 3B, the building-blocks strings/chains of the molecular strands/sequences may include respective population identification segments/sections ID-SEG (114) which include an identifying sequence of bases of a base-set compatible with the chemistry of the molecular sequences.

To this end, FIGS. 3A and 3B show two embodiments of the molecular data storage by which the molecules of different population can be arranged to reside together in a mixture, while enabling the separate and correct reading of the data encoded by each population. This each achieved by including a respective population identification segments/section ID-SEG (114) in each molecular strand/sequence of a population which is designed to reside in mixture with molecular strands of other population.

In the non-limiting embodiments shown in FIGS. 3A and 3B, the molecular strands/sequences include data encoding segments/section 115, which is configured similarly to that described above with reference to FIG. 1A. It should be understood that this embodiment is not limited to the configuration of the data encoding segments/section 115 of FIG. 1A, and that other configuration of the data encoding segments/section 115 may implemented in this embodiment, for instance the configuration of the data encoding segments/section 115 shown in FIG. 2B, as well as other configurations with any suitable values of the configuration parameters shown in Tables 2 or 6 of FIGS. 1B and 2C respectively. Accordingly, the above description about the configuration and operation of the data encoding segments/section 115 of FIGS. 1A and 2A is also valid for the embodiments of FIGS. 3A and 3B and will not be repeated here.

In the none-limiting embodiments shown in FIGS. 3A and 3B, the molecular strands/sequences also include respective population identification segments/sections ID-SEG (114) in the molecular sequences PM of each respective population 112, whereby the respective population identification segments/section in each molecular sequence/strand includes an identifying sequence of bases or building blocks identifying the respective population 112 to which the molecular sequence/strand belongs.

In the none-limiting examples of FIGS. 3A and 3B, the identification segments/sections ID-SEG (114) include an identifying sequence of bases of the same base-set, e.g. [A,C,G,T] which is used in the data encoding building-blocks. It should however be noted that although using the same base-set is practically preferable, it is not essential, and some implementations may use a different base set for the identification segment/section than the base set used of the data encoding segments (as long as these base-sets are chemically compatible in the same molecules).

The identifying sequence ID-SEG in the population identification segment 114 of each of the molecular strands/sequences PMs is indicative of the population 112, with which the respective molecular strand/sequence is associated, and is different in molecular strands/sequences of different data-blocks 110 (i.e. is different in molecular strands/sequences of different ones of said plurality of populations associated with the different data-blocks 110).

It should be noted that the population identification segments ID-SEG (114) generally do not code the letters of the alphabet Σ encoded by the data encoding sections 115. Indeed, the alphabet letters of the coded sequence S of letters are coded collectively by the plurality of molecular strands of each populations, whereby the population identification segments/sequence ID-SEG/114 is used to mark each individual molecule/strand of to indicate to which population it belongs (associated with). Accordingly, identification segments of the same ID similar in all the molecules marked thereby. In other words, the population identification sections/segments, which are unique identifiers of the respective population 112 (i.e. distinguishing the respective population from others), are encoded by a fixed sequence/order set of building-blocks or bases (e.g. typically consecutive ordered set—but not necessarily consecutive), which identifies the respective population. It should be understood that generally more than one different ordered set/sequence of building-blocks/bases may be used to identify same populations (so in some implementations some different molecular strand/sequences of the same population may be include different population identification segments all indicative of the same population to which the different molecules below). Nonetheless different molecular strand/sequences of the different populations are essentially marked by different identification segments indicative of their respective population so that the molecules of different populations are distinguishable in terms of their population, based on their ID-SEG (114)).

In the present example of FIG. 3A, the identification sequence/segment ID-SEG (114) of the molecules of the different populations is formed as a sequence of bases selected from the Sz number of bases [A,C,G,T]. For instance, the populations 112 which of the respective data-block 110.1, is marked/identified by the ordered set/sequence of the bases/monomers T-A-G in the identification segment ID-SEG (114) of the molecular strands/sequences PMs. To this end it would be appreciated that the locations in the ID-SEG (114) of the molecules in the figure are shown in terms of the molecular bases, while the locations in the data encoding sections 115 in this figure are shown in terms of the basic molecular building blocks (e.g. each occupying the length of k>1 bases, where here k=3).

In the example of FIG. 3B, the identification sequence/segment ID-SEG (114) of the molecules of the different populations is formed as a sequence of building blocks selected from the Z number of building blocks {E_{z}}_{z=1 to Z}. For instance, the population 112 which of the respective data-block 110.1, is marked/identified by the ordered set/sequence of the k-mer (trimer) building blocks E₁-E₂-E₂. Indeed as can be appreciated, in this example a somewhat longer identification sequences would be required in order to enable to distinguish between a given plurality of populations, as compared to the embodiment of FIG. 3A. This is because here the building blocks are used which are short k-mers satisfying certain prerequisites (e.g. k>1 and H1>1), and not any base sequences can be synthesized thereby. However, this embodiment is advantageous over the embodiment of FIG. 3A because it does not require the data storage synthesizing machine to be configured for synthesizing the molecules based on both building blocks {E_{z}}_{z=1 to Z} and the individual bases (e.g. [A,C,G,T]), as might be the case with the embodiment of FIG. 3A; and also because the use of the building blocks {E_{z}}_{z=1 to Z} for the identification segments provides some robustness to errors in the encoding of the data identification segments. To this end it would be appreciated in this embodiment the locations in the ID-SEG (114) of the molecules in the figure as well as the locations in the data encoding sections 115 in this figure are shown in terms of the basic molecular building blocks (e.g. each occupying the length of k>1 bases, where here k=3).

Referring back together to both embodiments of FIGS. 3A and 3B, as indicated above, in both these embodiments the identification section 115 included in the molecules, enables place the molecules of different populations in a mixture while still enabling to distinguish between them for separate accurate reading/sequencing of the data of each population. For instance, utilizing specifically designed binding molecules, the molecular strands/sequences PMs of the population 112 may be exclusively extracted from a collection/mixture of molecular strands/sequences PMs of several data-blocks 110 (of several populations {112}) and separately sequenced to read/infer the data of the respective data-block 110.1 to which they belong.

It should be noted in some embodiments, e.g. particularly in case where the molecular strands/sequences are composed of A,C,G,T bases/monomers, the identification segments can be located at the so called 5p-end of the molecules, or at the so called 3p-end of the molecules, or, generally they may also be located anywhere else along the monomer/building-block strings/sequences of the molecules. In some particular implementations/embodiments of the invention, it may be preferable to locate the identification segments on the 5p-end of the synthesized molecules. This is because the quality of synthesized polymer tends to be higher at the 5p-end of the molecule.

The tables in FIGS. 3A and 3B are similar to Table 1 in FIG. 1A and show the correspondence of the encoded sequences in the data segment/section 115 to the alphabet letters of Table 4 in FIG. 1D. It should be noted that in some embodiments of the present invention the molecular strands/sequences PMs of different populations/data-blocks 110 are configured/synthesized such that the identifying sequences ID-SEG (114) which identify different ones of the populations/data-blocks 110 differ from one another by a difference exceeding a certain predetermined threshold. More specifically, in some embodiments of the present invention, the molecular data storage 100 may be configured such that each two different identification sequences/segments of building-block/bases which are used for identifying molecular strands/sequences of different populations/data-blocks differ from one another by at least a certain predetermined hamming distance threshold H3. For example, the certain distance metric of strings used may be the so called edit distance (as generally known in the art), and the minimal threshold edit distance between different identification sequences/segments may be, in some cases, at least 3 edit operations measured in the edit distance metric. Using the certain minimal distance (e.g. H3=3) may be preferable because the mapping of the letters in the population to the composite alphabet depends on identifying every molecule as a member of the correct population.

Referring to FIGS. 1A and 2A, it is noted that the identifying sections 114 of the sequences are marked as optional in these embodiments. In this regards it should be understood that the inclusion of identification sequences 115 in the molecules is indeed optional an is required only in embodiments where more than one population is included in the data storage system and only in case where the molecules of several, two or more, populations of different data blocks are co-located (e.g. in a mixture). Alternatively, in embodiments where the molecules of different populations are arranged at separate locations/regions, there may be no need for including such identification sequences 115 in the molecules as distinction between molecules of different population may be based on their location/region.

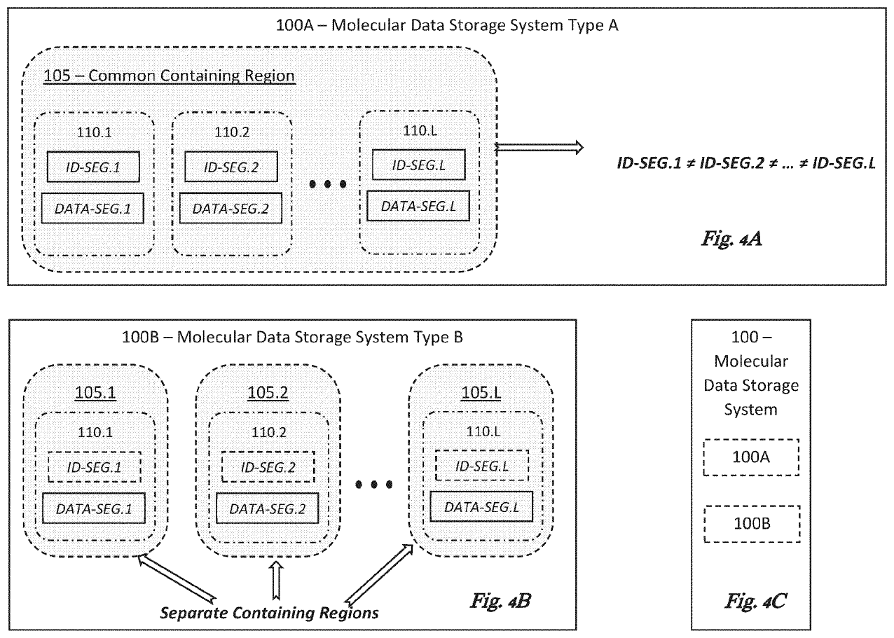

Turning now together to FIGS. 4A to 4C, these are block diagrams showing three types of molecular data storage systems according to various embodiments of the present invention. Systems 100A, 100B shown in FIGS. 4A and 4B, are two types of molecular data storage systems according to two embodiments of the present invention, in which the molecular strands/sequences of different populations are contained together (e.g. at the same region), and separately (e.g. at different respective regions), respectively; and system 100 shown in FIG. 4C is a generalized/generic system type whose configurations are combinations of the configurations shown in systems 100A 100B (namely some of the populations may reside together, e.g. in a mixture, while others may reside separately).

In the all the exemplified data storage systems, shown in FIGS. 4A to 4C, the plurality of L data-blocks 110.1, 110.2 . . . 110.L, include respective populations of molecules with respective data segments DATA-SEG.1, DATA-SEG.2 . . . DATA-SEG.L by which the data is encoded utilizing an alphabet Σ according to the present invention (e.g. such as that discussed above with references to FIGS. 1A to 2C).

In the molecular data storage systems type A, 100A, shown in FIG. 4A, molecular strands/sequences of the plurality of populations/data-blocks 110.1, 110.2 . . . 110.L are contained together in a common containing region 105. The molecular strands/sequences of each population/data-block, include the similar identification segment, e.g. molecules of data-block 110.1 are identified-by/include the unique id segment ID-SEG.1, molecules of data-block 110.2 are identified-by/include the unique id segment ID-SEG.2 and so forth, molecules of data-block 110.L are identified-by/include the unique id segment ID-SEG.L (the id segments differ from one another ID-SEG.1≠ID-SEG.2≠ . . . ≠ID-SEG.L). To this end, molecular strands/sequences PMs associated with the same population, can be exclusively selected by utilizing binding molecules configured and operable for selectively binding to the population identification segment of the molecular strand/sequence of the same population.

In the molecular data storage systems type B, 100B, shown in FIG. 4B, molecular strands/sequences of the plurality of populations/data-blocks 110.1, 110.2 . . . 110.L are contained/reside separately, in spatially separated respective regions 105.1, 105.2 . . . 105.L. In this case, the unique id segments ID-SEG.1, ID-SEG.2 . . . ID-SEG.L are only optional and may be obviated from the molecular strands/sequences since the molecular strands/sequences of different populations may be distinguishable based on the spatial location in the data storage 100B. To this end, the molecular data storage systems type B, 100B, may include a structure of a plurality of distinct regions 105.1, 105.2 . . . 105.L at which molecular strands/sequences of different respective populations reside respectively.

The general molecular data storage system 100 shown in FIG. 4C, combines the techniques of the molecular data storage systems types 100A and 100B, and may thus include some populations/data-blocks whose molecular strands/sequences are spatially separated as in type B systems (thus not necessitating identification segments in these molecules), and may also include some populations/data-blocks whose molecular strands/sequences are co-located at the same regions and thus have different identification segments which enable to distinguish between molecules of the different populations that reside together.

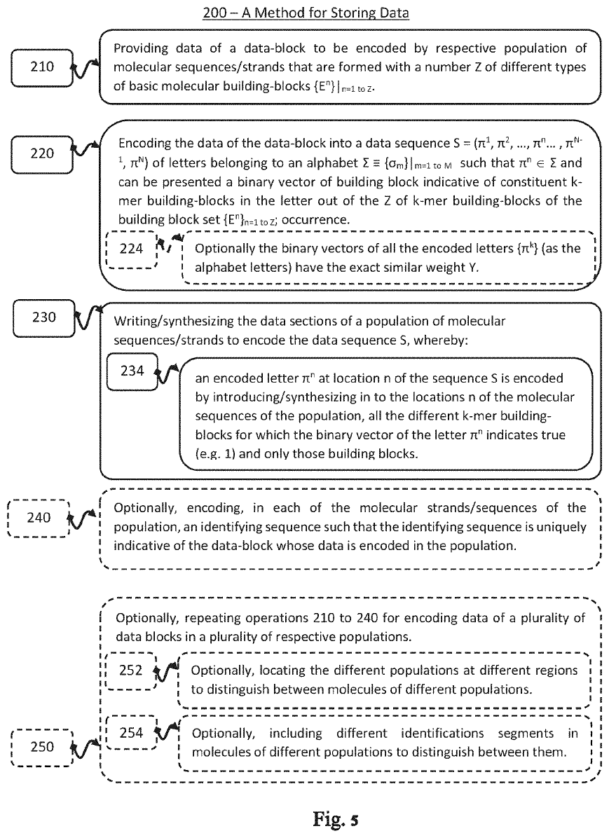

Reference is now made to FIG. 5 showing a flow chart of a method 200 for storing data according to an embodiment of the present invention. The method 200 may be implemented in conjunction with the molecular data storage system 100 described above of the present invention. The method includes the following:

In 210 data of at least one data-block (e.g. 110.1) to be stored by the system, is provided. The data is designated to be encoded by a respective population (e.g. 112) of molecular strands/sequences PMs that are formed with a number Z of different building-block types {E^{z}}_{z=1 to Z}. In 220 the data of the data-block 110.1 is processed for presenting it as data sequence S=(π¹, π², . . . , πⁿ . . . , π^{N-1}, π^{N}) of letters of the alphabet Σ≡{σₘ}|_{m=1 to M} which is used according to the present invention, as described above (namely the alphabet Σ in which each letter is represented as a unique binary vector over the space spanned by short k-mer building blocks with k>1, and possibly with certain prerequisite parameters such as Y, H1, H2). The alphabet Σ may be such as that represented in FIG. 1D or 2B, defined by respective binary vectors σₘ≡indicative of the constituent building-blocks/k-mers in each letter.

Optionally, as shown in 224, the binary vectors of all the encoded letters {πⁿ} (as well as the alphabet letters) have the exact similar weight (exact number parameter) Y as described above.

Step 230 includes writing/synthesizing the data encoding sections 115 of the population 112 of molecular strands/sequences, such that the data encoding sections 115 of the population 112 collectively encode the sequence S of encoded letters S=(π¹, π², . . . , πⁿ . . . , π^{N-1}, π^{N}). The synthesis of the data encoding sections 115 of the population 112 is conducted utilizing the data encoding building-blocks {E_{z}}_{z=1 to Z} as the basic elements of the synthesis (e.g. not base by base synthesis, but building block by building block synthesis). Such implementation increases the synthesis speed by up to k times as compared to base by base synthesis (which is another advantage of using k-mers with k>1 as the building blocks.

In 234, the sequences of encoded letters S=(π¹, π², . . . , πⁿ . . . , π^{N-1}, π^{N}) is synthesized. Each encoded letter πⁿ is written/synthesized by introducing/synthesizing all and only the short k-mer building-blocks for which the binary vector of the letter indicates true (e.g. 1), into the corresponding locations n of the plurality molecular sequences of the population. Accordingly each encoded letter πⁿ at location n of the sequence S is formed/corresponds-to/is indicated by the existence/occurrence or not of each building-block of the building blocks {E^{z}}_{z=1 to Z} the location n along the building-blocks strings of the plurality of molecular strands/sequences of the population 112. The encoded letter πⁿ corresponds to a respective alphabet letter σₘ∈Σ whose binary vector correctly represents the types of building-blocks existing/occurring at the location n of the encoded letter.

Optionally, as shown in 240, an identifying sequence of building-blocks or bases is synthesized in each of the plurality molecular sequences of the population. (As indicated above, the identifying sequence is selected to be unique per population so it may serve as a unique identifier of the data-block whose data is encoded in the population 112.

It should be understood that the order of the synthesis of operations 230 and 240 is not necessarily as depicted in the figure and may be different in different implementations of the invention. For instance, synthesis of the identification sequences may precede or proceed the synthesis of the data sequences of may be performed intermittently.

Optionally, as shown in 250, a plurality of data blocks with respective data are encoded/formed by repeating 210 to 240 to provide a respective plurality of populations that encode the corresponding data of the plurality of data blocks. As indicated in 252 the different populations may be located at different regions to enable to distinguish between molecules of different populations. Alternatively or additionally, as shown in 254 molecular strands/sequences of different populations include different identifications segments/sections identifying their respective population, and distinguishing between the molecules of different populations.

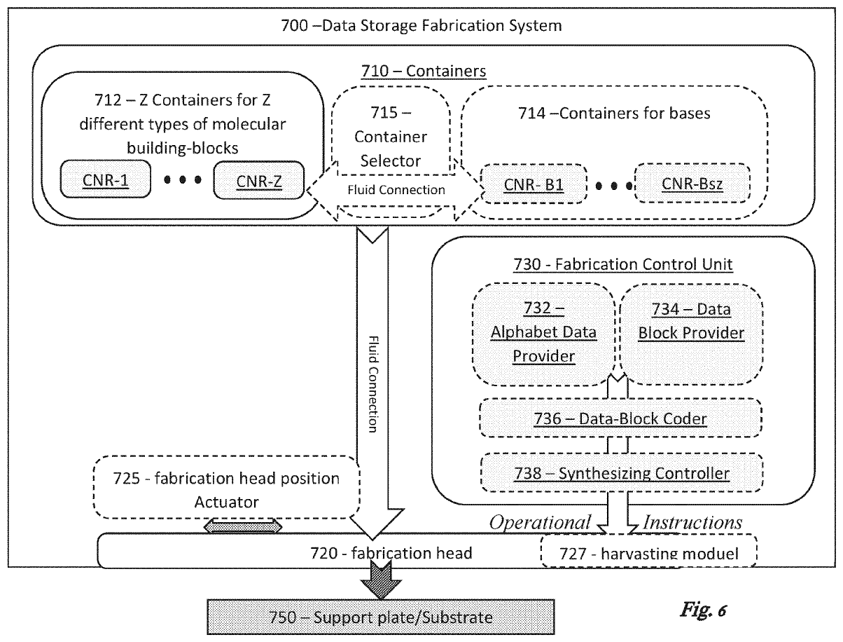

Reference is now made to FIG. 6 illustrating a block diagram of molecular data storage fabrication system 700 according to an embodiment of the present invention. The molecular data storage fabrication system 700 is configured and operable to fabricate a molecular data storage structure/system 100 such as those described above with references to FIGS. 1A to 4C.

According to some embodiments, the molecular data storage fabrication system 700 includes containers module 710 including building block containers 712. The building block containers 712 include at least Z building-block containers CNR-1 to CNR-Z, for containing respectively the Z types of short k-mer building-block {E^{z}}|_{z=1 to Z}, which are used for fabricating the molecular strands/sequences PMs of the molecular data storage system 100, or at least the data encoding sections 115 thereof. Preferably, the number Z of building block containers 712 does not exceed 50 (i.e. the alphabet is constructed with the number Z≤50).

Optionally, containers module 710 also includes bases' containers 714 including containers CNR-B1 to CNR-Bsz for containing individual bases (e.g. of the base set used to construct identification sections 114). This may be the case in embodiments where the fabrication system 700 is configured for fabricating a molecular data storage system 100 of the embodiment of FIG. 3A, wherein the molecular strands/sequences PMs of the molecular data storage system 100 include identification sections/segments 114 constructed as sequences of individual bases (not necessarily formed as sequence of the Z k-mer building blocks). The molecular data storage fabrication system 700 also includes a molecular strand/sequence fabrication head 720 that is fluidly connected to the container module 710, via a container selector 715 that is configured and operable to selectively fluidly connect one or more of the containers CNR-1 to CNR-Z, and optionally also one or more of the containers CNR-B1 to CNR-Bsz to the molecular strand/sequence fabrication head 720. The fabrication head 720 is configured and operable for selectable and controllable deposition of building-blocks, which are contained in a selected one of the Z building-block containers. In this sense, the fabrication head 720 may be configured and operable as k-mer printing jet head capable of injecting/depositing k-mer building-blocks from a selected container according to instructions provided to the fabrication head 720 from a fabrication control system/unit 730, which is also a part of the system 700. In embodiments including the containers of individual molecular bases CNR-B1 to CNR-Bsz, the fabrication head 720 may also receive instructions for, and is adapted to, inject individual bases from selected container of CNR-B1 to CNR-Bsz.

According to some embodiments of the present invention the fabrication control unit 730 is configured and operable to operate the fabrication head 720 and the container selector 715 for fabricating the molecular data storage system 100. To this end, the fabrication control unit 730 may include a Data Block Provider 734 configured and operable for receiving/providing at least one block of data (sequence S) that is to be encoded in the molecular data storage system 100. According to some embodiments of the invention, the data of the data block is encoded by “printing”/synthesizing a population of molecular strands/sequences at a region designated for the data block, on a support substrate/plate 750.

The fabrication control unit 730 may also include an alphabet Data Provider 732 which is adapted to provide (e.g. receive and/or retrieve from a reference data storage (e.g. local or remote memory) data indicative of an alphabet Σ, which is to be used for encoding the block of data on the designated location of the support substrate 750. To this end the block of data is to be synthesized/coded to encode in the population of molecular strands/sequences, a sequence of letters S={πⁿ}|_{n=1 to N} of the alphabet Σ≡{σₘ}|_{m=1 to M} whereby each encoded letter πⁿ at locations n along the molecular strands/sequences is represented as binary vectors (occurrence vectors) of Z different types of the data encoding molecular building blocks {Eₙ}|_{n=1 to Z} contained in the containers.

To this end, fabrication control unit 730 is adapted for synthesize the population of molecular sequences encoding said block of data at the designated region, by operating the fabrication head, to sequentially synthesize each letter πⁿ of the sequence S. In order to synthesize each letter πⁿ, fabrication head is operated to selectively deposit only the molecular building blocks indicated to be occurring by the binary vector representing the letter 70, from the Z containers.

As may be appreciated by those versed in the art, the fabrication head 720 may be configured similar to conventional molecular strands/sequences fabrication heads used for controlled synthesis of molecular strands/sequences. For instance see⁵. Also, according to some embodiments of the present invention, the types of basic building contained in the containers 710 blocks (and optionally also the bases is such are contained) are “blocked” (i.e. capped/protected; e.g. such as described in⁵) from one end thereof, in order to prevent their binding to one another. Accordingly, in some embodiments of the present invention the system 700 (e.g. the fabrication head 720) is configured and operable for carrying out the following after each deposition, at the designated region, of basic building blocks corresponding to each of the letters of the sequence S:

(a) Washing the region to remove un-bonded basic building blocks deposited at the region (this may be performed as conventionally done with molecular-strand/polymer synthesis⁵); and

(b) Applying un-blocking treatment to “un-block” (i.e. de-capping/de-protecting) basic building blocks from being bounded to molecules at the designated region (this may be performed as conventionally done with molecular-strand/polymer synthesis⁵).

Additionally, in some embodiments, the fabrication head 720 is configured and operable for depositing cleavable molecules at the designated region at which the population of the molecules should be synthesized. This is typically performed prior to the synthesizing. The system may also include a harvesting module 727 configured and operable for harvesting the population of molecules 112 from the designated region (e.g. by cleaving the cleavable molecules). The control unit may be adapted to operate the fabrication head 720 for depositing the cleavable molecules on the designated region of the substrate 750, prior to synthesis of the population of molecular strands/sequences. Then, synthesis of the population of molecular strands/sequences on the designated region such that they are bonded to the cleavable molecules is performed; then, after synthesis is completed, operating the harvesting module 727 for harvesting the population of molecules 112. Cleavage of molecules from surfaces that support the synthesis is described in the literature⁵.

As indicated above, in some embodiments the molecular strands/sequences of the population should include similar identification sections/segments (e.g. typically but not necessarily similar to all molecules of the population).

In some embodiments, e.g. see FIG. 3B, the identification segment/section includes an identifying sequence of the Z building-blocks/monomer types. Accordingly, the control unit 730 may be adapted for operating the fabrication head 720 for synthesizing the identification segment for all molecules of the population. This is achieved by drawing the building-block types from the Z building-block containers 712.

In some embodiments, e.g. see FIG. 3A, the similar identification segment/section includes an identifying sequence of molecular bases. In this case, containers module 710 also include the one or more additional containers, CNR-B1 to CNR-Bsz, for containing the individual molecular bases, the fabrication head 720 may also receive instructions for, and is adapted to, inject individual bases from selected container of CNR-B1 to CNR-Bsz. The control unit 730 is adapted for operating the fabrication head 720 to for synthesize the identification segment for all molecules of the population by drawing the molecular bases from the additional containers, CNR-B1 to CNR-Bsz.

In some embodiments the molecular data storage fabrication system 700 is configured and operable for fabricating different populations corresponding to different data-blocks 110 at different respective regions of the substrate 750. To this end the system may include a fabrication head position actuator 725 connectable to the fabrication head 720. The control unit 730 may be adapted for operating the fabrication head position actuator 725 for actuating/moving the fabrication head 720 to various designated regions on the substrate 750 and operating the fabrication head 720 to fabricate at each region a population of molecules corresponding to one of the plurality of data blocks. This provides for synthesizing a plurality of populations of molecular strands/sequences encoding data of a plurality of respective data blocks, at different spatially separated respective regions of the substrate 750.

It should be noted that in some embodiments, e.g. where harvesting is not performed, the molecular storage system 100 may actually be support plate/substrate 750 with the one or more populations of molecules thereon that were synthesized at the different regions thereof. Each population is associated with a respective data-block. Alternatively, or additionally, in some embodiments e.g. where harvesting is performed, the harvested populations may be placed in separate containers/containing-regions, or in a common container in case the molecules of each population can be exclusively identified by an ID segment included therein. In this case the molecular storage system 100 is actually implemented by the separate containers and/or the common container with the populations of molecules therein.

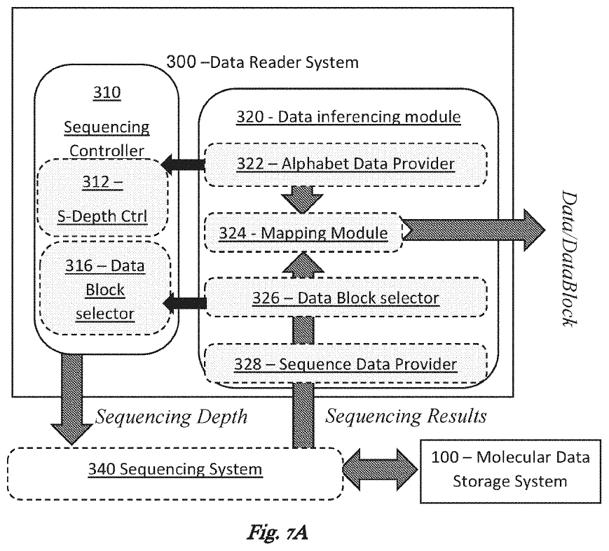

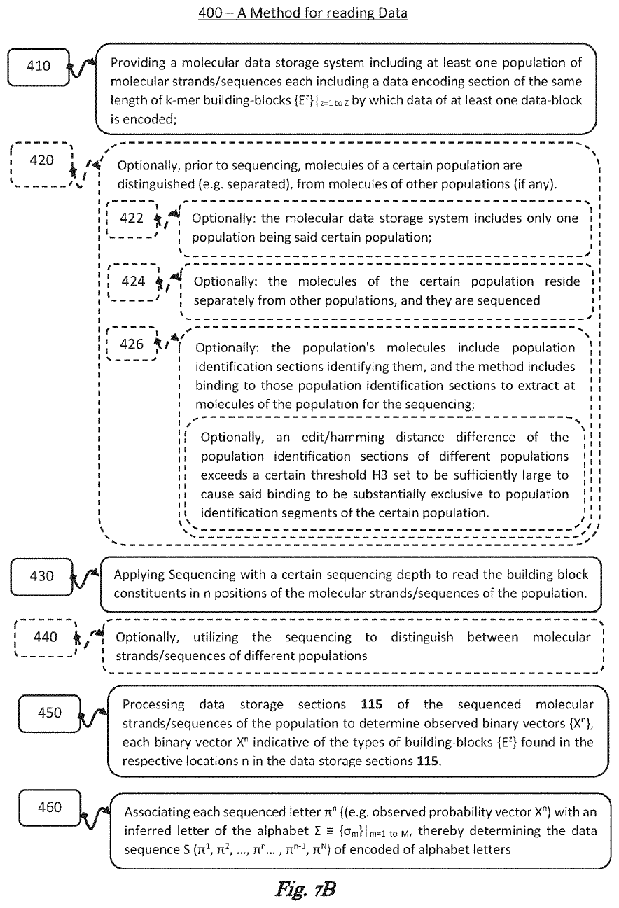

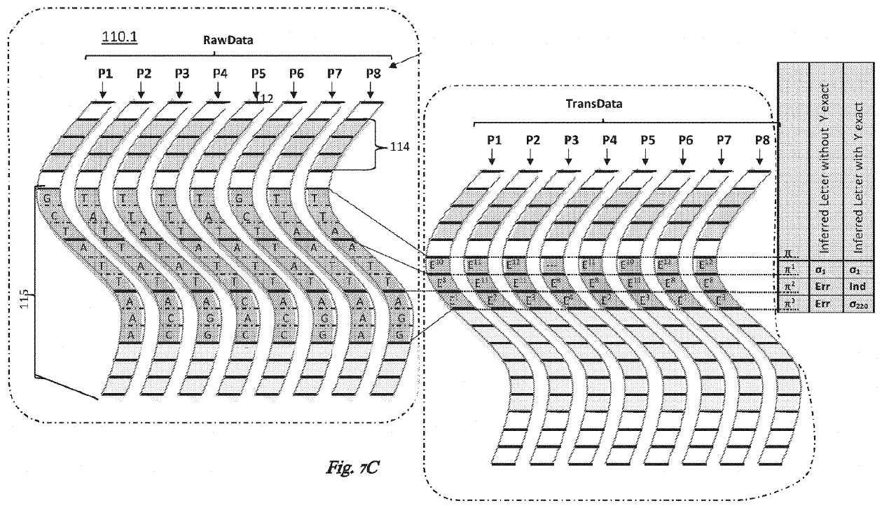

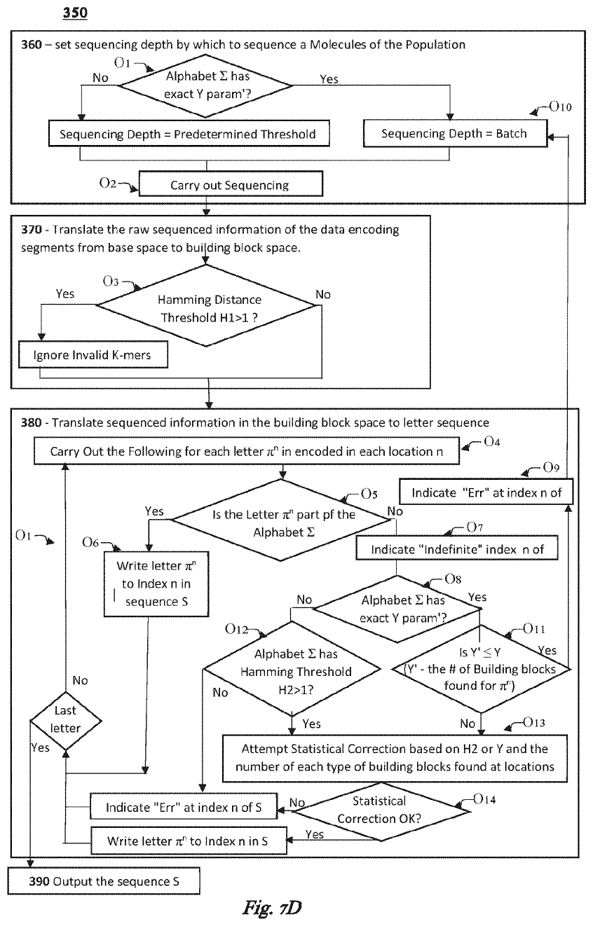

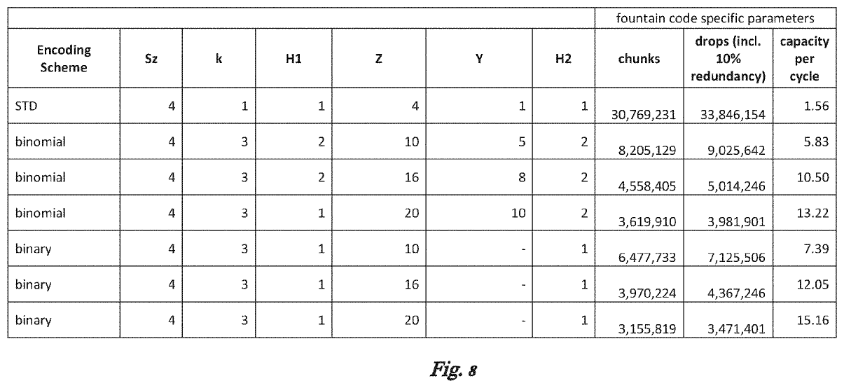

Reference is now made to FIG. 7A which is a block diagram of a data reader system 300 configured and operable according to an embodiment of the present invention. The data reader system 300 is configured and operable for reading/inferring data encoded in molecular data storage systems of the present invention, such as 100, 100A, 100B disclosed above with reference to FIGS. 1A to 4C.

The data reader system 300 includes a sequencing control module 310 (hereinafter also referred to as sequencing controller) configured and operable for connecting-to/communicating-with a sequencing system 340 (which may or may not be part of the system 300) for sequencing the molecules of a molecular data storage system 100, and data inferencing module 320 for processing the sequenced data (raw data) provided by the sequencing system 340 to determine the data stored by one or more data blocks.

It should be noted that the reading/sequencing process and accordingly the sequencing system 340 may be configured and operable according to any suitable sequencing technology, e.g. any NGS technology. In embodiments where nanopore sequencing technology is used, or other technologies with similar capabilities sequencing reads can be collected dynamically (e.g. on-line or in real time). Accordingly, in such embodiments, in case Binomial encoding scheme is used, a dynamic stopping condition may be employed, e.g. after observing all the Y included 3-mers at every position. This is because, the nanopore sequencing method/technology, or the similar technologies which allow the dynamic stopping condition, is capable of rejecting sequenced molecules as they are sequenced, which is useful in the reading process¹⁰. By identifying the sequenced molecules using the barcode/identification section, these techniques facilitate to selectively read, at any time point t, only molecules coming from populations for which lower coverage/lower-sequencing-depth was observed until time t. Thus, a desired sequencing depth can be achieved across all oligos in a consistent and uniform manner. The target sequencing depth R (i.e. the target coverage) can be set to ensure sufficiently low probability of errors in identifying all letters in the sequence S encoded by the population.

The sequencing control module 310 is adapted to operate the sequencing system 340, to sequence the molecules of one or more data blocks (e.g. 110.1) of a molecular data storage system 100 such as that of exemplified in FIGS. 1A to 4C, and the data inferencing module 320 includes a part that is adapted to communicate with the sequencing system 340 to receive the raw sequenced data, e.g. in terms of the sequenced molecular bases. The inferencing module 320 also includes an alphabet data provider 322 and a mapping module (mapper) 324. The alphabet data provider 322 is configured and operable for obtaining data indicative of an alphabet Σ which is used for encoding data in a molecular storage system of the invention (e.g. the alphabet data may be such as that described with reference to FIGS. 1A to 2C above, e.g. including data indicative of the short-mer building blocks, e.g. as in Tables 4 and 5). The mapping module (mapper) 324 is adapted to receive the alphabet Σ from the alphabet data provider 322 and the raw sequenced data from the Sequence Data Provider 328, and is configured and operable to process the raw sequenced data based on the alphabet Σ and infer the data stored in one or more data blocks (e.g. 110.1), while possibly utilizing the alphabet parameters such as H1, H2, and/or Y to apply error correction and/or data validation, and/or to dynamically adjust the sequencing depth, in order to enable data validation and/or error correction. To this end in case the alphabet Σ is based on the binomial encoding scheme (i.e. characterized by the exact fixed parameter Y where each letter is being specified by exactly Y different building blocks as in the embodiment of FIGS. 1A to 1E), the sequencing depth R can be dynamically adjusted during the data reading as inferred based on the sequenced raw data, or it can be set apriori with accordance to required probability of correct reading. Alternatively, in case the alphabet Σ is based on the binomial encoding scheme (i.e. does not implement the exact number parameter Y), the sequencing depth may be set/predetermined in advance however it might be difficult to determine to meet a certain sequencing depth threshold R to provide respective probability for correct reading.

More specifically, the following provides an analysis of probabilities of correct letter identification for alphabets defined according to the Binomial and Binary encoding schemes. In both cases embodiments where the Hamming distance H1≥2 are considered, i.e. in which the short k-mers serving as the data encoding building block set {E^{z}}_{z=1 to Z} are only a subset smaller than all possible k-mers of length k of bases. Accordingly the probability of reading mix-up errors between building blocks can be deduced by proper predetermined selection/adjustment of the Hamming distance threshold H1, which in turn also affects the size Z of the building block set {E_{z}} and the data density of the encoding. Assuming the Hamming distance threshold H1 is adjusted for near-zero probability for mix-ups errors (for reasonable values of k this may be achieved with H1>3), the remaining source of reading error for a data sequence S stored by a population of molecules is related to insufficient sampling (insufficient sequencing depth) of molecules of the population by which the data sequence is represented in the storage—leading to misinterpretation of the encoded letters of the data sequence S.

In general, considering R to be the sequencing depth/coverage for a given population storing a respective data sequence S of length N, the probability p(S) of correctly identifying the stored data sequence is determined as:

p(S)=Πₙ₌₁^{N}P(Sₙ)

where p (Sₙ) is the probability of correctly identifying the n-th encoded letter πⁿ of S. In the following, p (Sₙ) is estimated for the different encoding schemes discussed and exemplified above with reference to FIGS. 1A to 1D, and 2A to 2C respectively.

I. Binomial Encoding Scheme/Alphabet:

Since every letter σ∈Σ consists of the exactly Y included k-mers, the required number of reads to observe at least one read of each k-mer building block E_{z} follows the coupon collector distribution¹¹. The number of reads required to achieve this goal can be described as R=Σᵢ₌₁^{Y}Oᵢ where O₁=1 and

$${O}_{i} \sim Geom ( \frac{Y - i + 1}{Y} ) ,$$

i=2, . . . , Y. The expected required sequencing depth R (the expected number of required reads) is then:

$$E [ R ] = {\sum }_{i = 1}^{Y} E [ {O}_{i} ] = Y {\sum }_{i = 1}^{Y} \frac{1}{i}$$

and can be for example approximated by Y log(Y). The entire distribution is easy to calculate, using convolution, for design purposes. The number of reads required for observing all included k-mer building blocks is reasonable in this case even for large values of Y.

For reasonable values of Y (e.g. Y≤10), a standard sequencing depth/coverage of R=100 reads per population yields low probabilities to miss one of the included k-mer building blocks in each letter. Note that with an online sequencing technology (i.e. nanopore sequencing) we can simply keep sequencing until all k-mers have been observed. The above bounds then provide an estimate on the expected actual sequencing cost but there is no issue with reconstruction.

As can be appreciated by the above description, the probability of observing/reading an encoded letter πⁿ with a k-mer building block that is not included in the set of Y k-mers of the definition of the alphabet letter σₘ to which it corresponds is generally diminished by requiring the minimal Hamming Distance H1≥2 between the k-mer building blocks {E_{z}} of the building block set. However, in some embodiments of the present invention, further protection against this type of error is provided by a condition of observing (sequencing) each of the Y k-mer building blocks {E_{z}} of each encoded letter at least t>1 times/reads. The derivation of the number of required reads is not as simple in this case but can be approximated by Y(log(Y)+t log log(Y)). Thus also in this case a reasonable sequencing depth R can be obtained for relevant values of Y and t.

In this regards it is noted that in embodiments where the Binomial encoding scheme is implemented (i.e. embodiments with the exact parameter Y of the building blocks occurring in each letter, such as FIGS. 1A to 1D), there exists a simple stopping condition for the sequencing. Thus, in such embodiments, in case dynamic sequencing approach is conducted during the reading, apriori setting the sequencing depth R may be unnecessary. For example, utilizing the selective nanopore approach, the sequencing process may be carried out (e.g. dynamically) until the stopping condition that at least Y building blocks are read for each encoded letter. Accordingly reconstruction/reading issues associated with insufficient sequencing depth R may be avoided (as the sequencing depth is increased as needed until proper reading is obtained).

II. Binary Encoding Alphabet

In contrast to the Binomial encoding scheme (with the exact Y parameter), the binary encoding scheme (in which the exact Y parameter is not employed) does not allow for a simple stop condition since the number of k-mer building blocks included in the inferred letter is unknown. In such embodiments (e.g. as in FIGS. 2A to 2C), if t>1 is set to be the minimal (or desired) number of reads associated with each included k-mer building block, and R is the sequencing depth (the total number of observed reads) then the minimal probability p of observing less that t reads for any of the individual k-mer building blocks included in an encoded letter πⁿ is:

$$p = Probability of ( O \sim Binom ( \frac{1}{Z} , R ) < t )$$

where Binom is the Binomial Distribution function. This worst case is obtained when the true alphabet letter σₘ consists of all the Z k-mer building blocks.

Setting reasonable example values for Z=10, R=150 and t=2, the probability p is found to be

$$p = Probability of ( O \sim Binom ( \frac{1}{10} , 150 ) < 2 ) ≅ 2.5 \times {10}^{- 6}$$

Accordingly, as we expect millions of positions to be decoded for reasonable data encoded, sequencing with much higher sequencing depth R is required to be conducted in such embodiments (e.g. as compared to the embodiments with the Binomial encoding scheme), while providing reduced assurance of correct reading that the encoded letters of the sequence S.

In the above context, it is appreciated that a sequencing depth threshold R may be selected based on the alphabet and coding parameters, including whether binomial or binary scheme was used, so as to provide high statistical probability that the letters will be correctly inferred. Thus, the sequencing controller 310 may include a sequencing depth controller 312 configured and operable to adjust the sequencing depths. In case the alphabet Σ does not implement the exact number parameter Y, the sequencing depth controller 312 may be adapted to receive input/reference data indicative of an acceptable inference error iErr for the decoding process, and estimate the required sequencing depth threshold based on the acceptable inference error iErr and the alphabet characteristics, as depicted e.g. in Tables 2 and 6. In case the alphabet Σ does implement the exact fixed number parameter Y, the sequencing depth controller 312 may be adapted to initiate sequencing of batches of molecules (e.g. with predetermined sequencing batch depth at each cycle), and the mapping module (mapper) 324 may be connected to the sequencing depth controller 312 for dynamically initiating sequencing of additional batches of molecules in case it determines additional sequencing might facilitate error correction and/or data validation.

Reference is now made to FIG. 7B which is a flow diagram of a method 400 for reading data stored in a molecular data storage system according to an embodiment of the present invention. In some embodiments the data reader system 300 is adapted to implement method 400.

In 410, a molecular data storage system 100 including at least one data-block encoding data, e.g. 110.1, is provided. The at least one data-block 110.1 is formed by at least one respective population 112 of molecular strands/sequences PMs, which are comprise data encoding sections representing chains of short-mers which serve as data encoding building blocks according to the present invention, and belong to the building-block-set {E^{z}}|_{z=1 to Z} consisting of the number Z of different preselected short-mers by which data of the data-block is encoded (each data encoding building blocks is a unique/distinguishable combination of a similar number of predetermined k≥2 (plurality) of molecular bases. The data of the data-block 110.1 is encoded in sequence S′=(π¹, π², . . . , πⁿ . . . , π^{N-1}, π^{N}) (e.g. ordered) of encoded letters {πⁿ} belonging to the alphabet Σ, whereby the identity of each encoded letter πⁿ∈Σ is indicated by the types of building-blocks existing at certain respective locations corresponding to k along the building-block strings of the molecular strands/sequences of the population 112.

Optionally, in 420 (which may be carried out prior to sequencing of molecules of the population 112), the molecules of the certain population 112 may be distinguished (e.g. separated and/or identified), from molecules of other populations, if such exist. In case there is only one population/data-block, this operation is trivial, as shown in optional 422. Alternatively, or additionally, in case the data storage system 100 is configured such that the molecules of the certain population 112 reside separately from other populations, location-based sequencing 424 of the molecules may be performed only at the region of the population 112 thereby not sequencing (distinguishing from) molecules of other populations. Yet, alternatively or additionally, in case the molecules of the certain population 112 include population identification segments ID-SEG uniquely identifying the certain population 112, specific binding to these population identification segments 426 may be carried out in order to distinguish (exclusively extract) molecular strands/sequences of the certain population 112, for further sequencing (as indicated above, optionally the difference between the population identification sections of different populations, is sufficiently large to avoid binding errors).

In 430, sequencing is performed to the molecular strands/sequences of the data storage system 100, or just to the molecular strands/sequences of the specific/certain population 112 (depending on implementation).

Optionally, after sequencing 430, 440 is performed to distinguish between molecular strands/sequences of different population, based on the sequenced identification sections of the molecules. Operation 440 may be performed in cases where in cases where the molecules of more than one population 112 (more than one data block 110) are sequenced together.

In 450, the data storage sections 115 of the sequenced molecular strands/sequences PMs of the certain population 112 are processed to determine per each location n (of the N locations in the data storage sections 115), an observed binary vector Xⁿ indicative of occurrence/existence of any one of the Z types of building-blocks {E^{z}} at the locations n in the data storage sections 115. To this end, per each binary component indexed z of the 1 to Z binary components of the observed binary vector Xⁿ is indicative of whether a corresponding building block E^{z} of the building-block-set {E^{z}}|_{z=1 to Z} was found/sequenced at the location n corresponding to the index of the binary vector Xⁿ along any of the sequenced molecular sequences/strands of said population.

In 460 the sequence S of letters encoded by the population 112 of the read data block (e.g. 110.1), are inferred, by associating each observed binary vector Xⁿ (each sequenced letter πⁿ with a inferred letter σⁿ of the alphabet Σ≡{σₘ}_{m=1 to M}. This may include mapping the observed binary vector Xⁿ at each location n to one of the letters {σₘ}|_{m=1 to M} of the alphabet Σ by determining a match between the observed binary vector Xⁿ and the binary vector definition of the letters. The technique according to which such mapping may be implemented according to some embodiments of the present invention, in order to suppress various errors (e.g. synthesis errors; degradation errors and/or sequencing errors) is described in more details below with reference to FIGS. 7C and 7D.

To this end errors may be suppressed and/or data may be validated by exploiting one or more of the following parameters of the alphabet as described above:
In case the building-block-set are characterized by having a certain first H1 threshold of minimal hamming distance between them H1≥2—this may be exploited to ignore sequenced short-mers that do not belong to the building block set—thus avoiding minor sequencing errors; and/or degradation errors by which one (or possibly more) base is replaced by another.
In case the alphabet Σ≡{σₘ}|_{m=1 to M} consists only of binary vectors with hamming distances between them greater or equal to a certain second threshold of minimal hamming distance H2≥2—any match t match between an observed binary vector Xⁿ and one of the letters {σₘ}|_{m=1 to M} of the alphabet Σ may considered valid/correct (since any wrong letter is distanced at least H2≥2 building blocks from that matched—i.e. at least H2 building block errors would be required to erroneously infer a different letter).

In embodiments where the alphabet letters Σ≡{σₘ}|_{m=1 to M}, are defined by occurrence of a predetermined exact number Y of the different types building-blocks {E^{z}}, sequencing with dynamic sequencing depth may be implemented. Sequencing may be continued until per each location n of the 1 to N locations in the data encoding sections, at least the exact number Y of different types building-blocks {E^{z}} is found/sequenced.

In embodiments where the alphabet letters Σ≡{σₘ}|_{m=1 to M}, are defined by the exact number Y parameter an improved data reading validation/correction operation may be carried out as follows, per each location n of the 1 to N locations of the data encoding sections:
(i) in case a weight Y′ of said observed binary vector Xⁿ is equal to the exact number parameter Y—the encoded letter πⁿ at the location n may be determined by mapping the observed binary vector Xⁿ to one of the alphabet letters {σₘ}|_{m=1 to M} (e.g. based on a match between the observed binary vector Xⁿ and binary vector representations the alphabet letters).
(ii) in case a weight Y′ of said observed binary vector Xⁿ is larger than the exact number parameter Y—it indicates that an excess Y′−Y of different types of building blocks is found at the locations n of the data encoding sections. In this case statistical significance of each of the Y different types of building blocks found at location n may be computed based on a number of time each of the Y′ types of building blocks is sequenced from the locations n:
   in case there are less than Y′−Y types of Y′ building blocks whose statistical significance are below the predetermined statistical significance threshold ST, determining that the observed binary vector Xⁿ may not be mapped to any one of the alphabet letters {σₘ}|_{m=1 to M} and thereby the encoded alphabet letters πⁿ at the location n is invalid;
   in case statistical significance of Y′−Y types of said Y′ building blocks are below a predetermined statistical significance threshold ST carrying out the following:
      determining that said excess Y′−Y types of building blocks are read in err, and amend the observed binary vector Xⁿ accordingly to obtain an amended observed binary vector X′ⁿ of weight Y; and
      determining the encoded alphabet letters πⁿ at the location n by mapping the amended observed binary vector X′ⁿ to one of the alphabet letters {σₘ}|_{m=1 to M} based on a match between the amended observed binary vector X′ⁿ and a binary vector representations of the alphabet letters;
(iii) in case a weight Y′ of the observed binary vector Xⁿ is less than the exact number parameter Y, determining that the observed binary vector Xⁿ may not be mapped to any one of the alphabet letters {am}|_{m=1 to M} and thereby the encoded alphabet letters πⁿ at the location n is invalid.

In this regard, reference is now made to FIGS. 7C and 7D. FIG. 7C shows the raw sequenced data RawData of a data block 110.1 in terms of the sequenced molecular bases, the translated sequenced data TransData presented in terms of the k-mer building blocks of the alphabet, and the table showing the letters inferred from the sequencing. the sequenced data in FIG. 7C exemplifies the result of sequencing depth of value 8 of a population of molecules of the data-block 110.1 in FIG. 1A (with the Alphabet parameters of this figure). For clarity in this none-limiting example, only 8 molecules P1 to P8 of the population 112 of data-block 110.1 are sequenced, and the figure depicts only the bases/short-mer building blocks of 3 sequenced letters π¹ to π³. FIG. 7D is a flowchart provided in self-explanatory manner for exemplifying a method of operation 350 of the data reader system 300 (and more specifically of the mapping module (mapper) 324 and the sequencing depth controller 312) according to various embodiments of the present invention.

Operation 360 of the method 350 is typically carried out by the sequencing depth controller 312 and include determining/setting the sequencing depth by which to sequence the molecules of the population 112 of the data block which is to be read, and operating the sequencing system accordingly. In this regards in operation O1 the sequencing depth is set. As indicated above, in case the alphabet implements the exact number parameter Y, the sequencing depth may increase dynamically during the processing and thus initial sequencing depth be set to certain batch value (e.g. the minimal/optimal value which may expected to be sufficient). In case it would be apparent that the sequencing is not sufficient, sequencing of another batch may be carried out, as explained below. Alternatively, in case the alphabet does not implement the exact number parameter Y an sequencing depth is selected according to a preset threshold (e.g. the threshold may be computed or apriori selected according to the alphabet parameters and acceptable inference error rate). Then, in O2, sequencing of molecules of the population 112 with the selected sequencing depth is conducted and the results thereof, the raw sequenced data RawData as exemplified in FIG. 7C, is provided to the inferencing module 320.

Operation 370 of the method 350 is typically carried out by the inferencing module 320 (e.g. by the mapping module 324) in order to translate the sequenced raw data of the data encoding sections 115, from the representation in molecular base space to representation in the space of building blocks {E^{z}}. Here, some correction may be applied to the sequenced data, as shown in 03, in case the alphabet is characterized by a minimal hamming distance threshold H1>1 between the k-mer building blocks {E^{z}}. In this case invalid short k-mers, which are not building blocks may be ignored, so as not to wrongly interpret the data. This is illustrated in FIG. 7C, showing that for the letter al the molecule P3 shown in the RawData encodes the k-mer “T-T-A” at the corresponding location n=1 is not a valid building block in this case (see e.g. Table 3 in FIG. 1C). Accordingly, upon translation of the RawData to the building block representation, this k-mer in P3 is ignored “T-T-A” and is marked absent from P3 in the translated data See TransData in FIG. 7C. Thus, at the end of operation 370 each sequenced letter πⁿ from location n in the sequenced molecules, may be represented as binary vector Xⁿ of size Z in which each of the 1 to Z binary vector components, indexed z, is indicative of whether a corresponding building block E^{z} of the building-block-set {E^{z}}|_{z=1 to Z} was found/sequenced at the location n corresponding to the index along any of the sequenced molecular sequences/strands of the population. For example, the sequenced letters π¹ to π³ in FIG. 7C may be represented by the following binary vectors X¹ to X³ (e.g. in case the alphabet of FIG. 1C is considered): π¹→X¹=(0,0,0,0,0,0,0,0,0,1,1,1); π²→X²=(0,0,0,0,0,0,0,1,0,0,1,0); π³→X²=(1,1,1,1,0,0,0,0,0,0,0,0).

Operation 380 of the method 350 is typically also carried out by the inferencing module 320 (e.g. by the mapping module 324) in order to translate the sequenced information in the building block space to letter sequence. As indicated in O4, the following operations are carried out for each of the letters which need to be read, e.g. sequenced letters π¹ to π³ in FIG. 7C which are read from the sequenced data at respective locations n=1 to 3. In O5 it is determined if a sequenced letter π³ is part of the alphabet. Σ. For this the definition of the letters alphabet in terms of the building blocks (e.g. as shown in table, as shown e.g. in Tables 3 and/or 4 is used. The valid blocks found at each location n in the sequenced molecules (see Trans Data in FIG. 7C) are matched with the binary vector definition of the letters, and if there is a match it is determined that the sequenced letter πⁿ belongs to the alphabet and the location n in sequence S of read letters is updated according (see 06). For instance, the sequenced letter π¹ in the example of FIG. 7C is matching the alphabet letter al according to the alphabet definition (e.g. Table 3) and is this registered as such in the read sequence S. In case there is no match, the letter in location n of the sequence may be indicated as indefinite (see 07) and the processing may continue to try and correctly infer the letter, according to the parameters of the alphabet. This is the case for the sequence letters π² and π³ in FIG. 7C. In that case where the sequenced letter πⁿ does not belong to the alphabet, the processing may commence differently according to whether the alphabet implements the exact number parameter Y or not—see 08. In case the alphabet implements the exact number parameter Y, it is determined whether the weight Y′ of the sequenced letter is smaller or equal to the exact number parameter Y (see O11), and in that case further sequencing of additional molecule may enable to resolve the correct encoded letter. Accordingly, in this case the inference process may be sopped/halted and sequencing of additional batch of molecules is conducted, and in the meantime, Error may be indicated in location n of the sequence S indicated as indefinite (see O9 and O10). Sequencing of additional batches of molecules may be repeated one or more times until allow the sequenced letters are correctly inferred, or the all the molecular sequences of the population are sequenced, or a certain maximal sequencing threshold is arrived. In case where in O11 it is found that the weight Y′ of the sequenced letter is larger than exact number parameter Y, this means that more types of building blocks than should have being included in the letter are found at the respective location n of the sequenced letter. Accordingly statistical correction O13 may be applied in this case in order to determine which detected/sequenced building blocks types should be ignored (e.g. based on the number of times each type of building block of the building blocks {E_{z}} is encounter at the location n of the sequenced molecules (—e.g. neglecting the building block types which were encountered the least number of times in case the number of times there are found is statistically insignificant). This is the case which the exemplified sequenced letter π³ in FIG. 7C. in this example the exact number parameter Y is 3, however for the letter π³ the following 4 building block types are encounter/found: E¹ two times; E² two times; E³ three times and E⁴ one time. In this simplified example E⁴ is neglected since it was encountered only once (e.g. considering that this is insignificant relative to the number of times the other Y building blocks were encountered). Accordingly, the sequenced letter π³ is determined/assessed to be the alphabet letter σ₂₂₀. It is noted that making such a correction is made possible because the alphabet has the exact Y parameter. Otherwise, some similar correction might also be possible in some case if the alphabet has a hamming distance threshold parameter H2>1 between the letters, and only in case the number of building blocks types which are error is less than H2—see e.g. O12 and O13. In that case the fact that there are less than H2 err building block types may be recognized due to the mismatch between the sequenced letter and the letters of the alphabet, and these types of building blocks which are error may be neglected by the similar statistical correction indicated above in relation to O13. Thus, see O14, in case the statistical correction succeeds (e.g. is possible according to the statistical significances indicated above), the correct letter may be written to the sequence S or otherwise reading error may be indicated for that letter in the location n of sequence S. It is further noted that the exact number parameter Y of the alphabet facilitates to recognize reading errors in cases where some encoded building blocks of letter are not sequenced (as Y′ would be smaller than Y″). However, in cases where the alphabet does not implement the exact number parameter, the sequenced letter may be wrongly attributed to an incorrect alphabet letter (see e.g. letter π² in FIG. 7C). Finally, after all of the letters in the sequence S are inferred, the sequence S is outputted.

As illustrated in FIG. 7D, with the parameter Y, sequencing depth may be increased dynamically during the reading process as needed until all possible letters are properly read/inferred. This provides for both improved error correction, and improves the nominal reading speed (as no harsh sequencing depths minimum needs to be apriority determined). Additionally, the Hamming distance thresholds H1 and H2 also facilitate for correcting various errors during the reading.

Turning back to FIG. 7A, in order to selectively read the data of a specific data block, e.g. 110.1, in some embodiments the data reader system 300 may include the one or both the data-block selector modules 316 or 326. Data-block selector modules 316 is a part of the sequencing control module 310 and is adapted to operate the sequencing system 340, to sequence the molecules of specifically selected one or more data blocks (e.g. 110.1). Data-block selector modules 316 enables a priory selection (prior to sequencing) of the data block(s) e.g. 110.1 whose molecules need to be sequenced. Data-block selector modules 326 is a part of the data inferencing module 320 sequencing control module 310 and provides post priori selection of the data block(s) e.g. 110.1 whose data needs to be inferred (e.g. the sequencing may be carried out on a plurality of data-blocks 110 and the selection of which data block to infer may be performed after the sequencing is performed).

In this regard it should be noted that in various embodiments of the present invention the sequencing controller 310 may be adapted to operate the sequencing system 340 to sequence the plurality of populations (data blocks) of the data storage system 100, and the resulted sequenced data of the plurality of data blocks may be provided (e.g. from the sequencing system 340) to the Sequence Data Provider module 328 of the data inferencing module 320. In turn, the data inferencing module 320 may include the data-block selector module 326, configured and operable for selecting the one or more data blocks (e.g. 110.1) of the data storage system 100 whose data are to be determined/inferred, and extracting, from the sequenced data (sequencing results) which are received by the Sequence Data Provider module 328, the relevant sequencing data of the data of the selected one or more data blocks (e.g. 110.1).

Alternatively, or additionally, in some embodiments the sequencing controller 310 may be adapted to operate the sequencing system 340 to sequence the population(s) of only the selected one data block (or more than one data blocks) of the data storage system 100. The sequencing controller 310 may include a data-block selector module 316 that is configured and operable for selecting the data block (or the plurality thereof) which needs to be sequenced. This may be based on input data indicative of the required blocks. In turn, the sequencing system 340 operates to discriminate between (e.g. exclusively sequence) the molecular strands/sequences of the selected data block/population, whereby such discrimination may be based on the region/location at which the molecular strands/sequences of the selected data block/population are located in the data storage system 100 (i.e. considering that this location may be exclusive to the selected population) or by utilizing specifically selected binding molecules which are configured and operable to selectively bind to a unique identification segment associated with molecules belonging to the selected population. It should be understood that this technique can only be operated with populations whose molecules include respective identification segments, and only in case the case the sequencing system 340 includes (or can synthesize “on the fly”) one or more collections of binding molecules, where binding molecules of each collection are adapted to exclusively bind to a respective population (to the identification segment thereof). Thus, in that case, upon receiving operational instructions of the selected data block from the data-block selector module 316, the sequencing system 340 utilizes the designated region of the selected data-block/population, and/or utilizes/synthesized binding molecules capable of binding to the identification segment of the selected data-block/population, to extract/sequence the molecules of the selected data-block separately and provide the sequenced data/results to the Sequence Data Provider module 328.

In turn, regardless of whether data-block selector module 316 and/or data-block selector module 326 is used, the sequencing data/results corresponding to the data segments of the population of molecules in the selected data blocks are provided (separately per each respective data block) to the mapping module 324 of the data inferencing module 320.

Referring to FIG. 8, the table in the figure exemplifies the information capacities for selected encodings schemes utilizing Z≥2 shortmers (binary or binomial schemes) as compared with the standard encoding utilizing the molecular bases themselves (in all the examples molecules are constructed of Sz=4 bases, such as the DNA bases [A,G,C,T] are considered. More specifically, the encoding of a 1 GB input file using six different alphabets with 3 different 3-mer sets (of sizes 10, 16 and 20) and using two encoding methods—binomial and binary. All calculation are based on similar error correction schemes (e.g. ^{3,12}). Using these selected alphabets, the encoding up to 9.7-fold in information capacity per synthesis cycle; and 3.2-fold increase per molecular base (e.g. DNA base), as compared to standard, base by base data encoding techniques. All the examples in the table are computed considering the use of similar overhead of molecular lengths for data error correction codes, such as Reed-Solomon and barcode/identification-sections' lengths.

For example, with the Binary encoding scheme, using Z=10, the alphabet size is |Σ|=2¹⁰−1=1023 and every 19 bits can be encoded into a pair of letters from Σ. This results, after taking overhead into account, in information capacity of 7.39 bits per synthesis cycle (2.46 bits per base). Using this encoding scheme one can encode a 2.12 MB zip file using ˜15K molecules/oligos of 456 bp (i.e. 152 synthesis cycles) compared to 72K oligos using standard DNA based storage³. Note that we used blocks of 2 letters in this encoding. When using single letter blocks we get a capacity of 7 bits per cycle (2.33 bits per base).

With the Binomial encoding scheme, using Z=10 and Y=5, an alphabet of size

$$\vert \sum \vert = ( \begin{matrix}10 \\ 5\end{matrix} ) = 252$$

is obtained. Accordingly, every 15 bits can be encoded into a pair of letters from Σ. This results in information capacity of 5.83 bits per synthesis cycle (1.94 bits per base). Using this encoding scheme a 2.12 MB zip file can be encoded using only ˜19K molecules/oligos of 456 bp (i.e. 152 synthesis cycles) compared to 72K oligos using standard DNA based storage³. Note that we used blocks of 2 letters in this encoding. When using single letter blocks we get a capacity of 5.44 bits per cycle (1.81 bits per base). Using Z=16 and Y=8, an alphabet of size

$$\vert \sum \vert = ( \begin{matrix}16 \\ 8\end{matrix} ) = 12 , 870$$

is obtained, which can encode every 27 bits into a pair of letters from Σ. This results in information capacity of 10.5 bits per synthesis cycle (3.5 bits per base). Using this encoding scheme one can encode a 2.12 MB zip file using ˜10.63K molecules/oligos of 456 bp (i.e. 152 synthesis cycles) compared to 72K oligos using standard DNA based storage³. Note that we used blocks of 2 letters in this encoding. When using single letter blocks, we get a capacity of 10.11 bits per cycle (3.37 bits per base).

1. A molecular data storage system for encoding one or more data-blocks, the molecular data storage system comprising one or more populations of molecular sequences, each population of molecular sequences encoding a respective data-block of the one or more data-blocks;
wherein each molecular sequence of the molecular sequences of the population comprises a data encoding section comprising a sequence of similar predetermined length N of short k-mers, whereby in each population the data encoding sections of all molecular sequences have the similar predetermined length N;
wherein said short k-mers serve as data encoding building blocks of the data encoding sections, whereby valid short k-mers serving as data encoding building blocks form a subset of a building-block-set consisting of a number Z of different preselected short k-mers each presenting a unique combination of a number k of bases of a preselected set of bases, characterized in that all the Z types of short k-mers in said building-block-set have a similar predetermined size k≥2 (plurality) of bases;
wherein the data encoding sections of the molecular sequences of the population collectively encode a sequence of encoded alphabet letters S=(π¹, π², . . . , πⁿ . . . , π^{N-1}, π^{N}); and
wherein each valid encoded alphabet letter πⁿ at location n of the sequence S of alphabet letters is characterized by occurrence of a predetermined plurality of different types of short k-mers of the building-block-set in a corresponding location n along the data encoding sections of the plurality of molecular sequences of said population.

2. The molecular data storage system of claim 1 wherein each valid encoded alphabet letter πⁿ at location n of the sequence S of alphabet letters is further characterized by occurrence of a predetermined exact number Y of the different types of short k-mers of the building-block-set in said corresponding location n in the data encoding sections, said predetermined exact number Y being the same for all the valid encoded alphabet letters; thereby enabling robust and efficient sequencing protocol by validating a letter encoded at said location n based on equality between said predetermined exact number Y and an actual number of Y′ of different types of short k-mers observed at said corresponding location n of said data encoding sections.

3. The molecular data storage system of claim 1 characterized in that all the different types of preselected short k-mers in said building-block-set have a similar predetermined size k≤20 of bases, thereby facilitating production scale data storage via molecular synthesis and low physical density.

4. (canceled)

5. The molecular data storage system of claim 1 wherein the Z different types of short k-mers in said building-block-set are characterized in that a hamming distance between each short k-mer in said building-block-set and any other short k-mer in said building-block-set is greater or equal to a certain first H1 threshold of minimal hamming distance, whereby said first threshold satisfies H1≥2, thereby enabling robust reading with error correction.

6. (canceled)

7. The molecular data storage system of claim 1 wherein each valid encoded alphabet letter to of the sequence S=(π¹, π², . . . , πⁿ . . . , π^{N-1}, π^{N}) belongs to a set of predefined alphabet letters Σ≡{σₘ}|_{m=1 to M} defined as binary occurrence vectors over the space spanned by said Z different types of short k-mer building blocks.

8. The molecular data storage system of claim 7 wherein said set of predefined alphabet letters Σ≡{σₘ}|_{m=1 to M} consists only of binary occurrence vectors of said space; and wherein at least one of the following:
said binary occurrence vectors are of equal weight;
said binary occurrence vectors having hamming distances between them greater or equal to a certain second threshold H2 of minimal hamming distance wherein said second threshold H2 of minimal hamming distance is at least (H2≥2).

9-12. (canceled)

13. The molecular data storage system of claim 1 wherein each molecular sequence of the molecular sequences of the population includes a population identification section comprising an identifying sequence of molecular bases indicative of the population with which said molecular sequence is associated; and wherein said identifying sequence is different in molecular sequences associated with different ones of said one or more populations.

14. The molecular data storage system of claim 13 wherein at least one of the following
the molecular bases included in said population identification section are bases of the same preselected set of bases by which said building-blocks are constructed;
the population identification section comprises an identifying sequence of said building-blocks;
a difference between the identifying sequences that are used in the population identification sections of different respective populations exceeds a predetermined threshold measured by a certain predetermined distance metric of strings, such as an edit or Hamming distance metric between strings.

15-16. (canceled)

17. The molecular data storage system of claim 13 wherein the molecular sequences of one or more of said populations are contained together in a common region; and wherein the molecular sequences associated with the same population can be exclusively selected by utilizing binding molecules configured and operable for selectively binding to the population identification section of the molecular sequences associated with said same population.

18-19. (canceled)

20. A method for reading data stored in a molecular data storage system, the method comprising:
(i) providing a molecular data storage system comprising a population of molecular sequences defining a data-block of the system;
(ii) applying sequencing to the population of molecular sequences and determining, per each location n of 1 to N locations in the data encoding sections of sequenced molecular sequences/of said population, an observed binary vector Xⁿ of dimension Z, whereby each binary component indexed z of 1 to Z binary components of the observed binary vector Xⁿ is indicative of whether a corresponding building block E^{z} of a building-block-set {E^{z}}|_{z=1 to Z} was sequenced at the location n corresponding to the index of said binary vector Xⁿ along any of the sequenced molecular sequences of said population;
wherein said molecular sequences of the population of said molecular data storage system comprise respective data encoding sections of similar predetermined length N of short k-mers serving as data encoding building blocks and forming a building-block-set {E^{z}}|_{z=1 to Z} consisting of a number Z of different preselected short k-mers by which data of the data-block is encoded, whereby each data encoding building block is a unique combination of a number k of bases of a preselected set of bases and wherein all the Z types of short k-mers in said building-block-set have a similar predetermined size k≥2 of bases; and
wherein the method further comprises:
(iii) determining encoded alphabet letters πⁿ of a sequence S=(π¹, π², . . . , πⁿ . . . π^{N-1}, π^{N}) encoded by said n=1 to N locations by associating each observed binary vector Xⁿ of each of said n=1 to N locations, to one of alphabet letters {σₘ} of a predetermined alphabet Σ≡{σₘ}|_{m=1 to M}; whereby each letter σₘ of the alphabet Σ is defined by a binary occurrence vector of size Z indicative of an occurrence of building blocks of said building-block-set {E^{z}} in the letter; said associating comprises mapping the observed binary vector Xⁿ at each location n to one of the letters {σₘ}|_{m=1 to M} of the alphabet Σ by determining a match between the observed binary vector Xⁿ and the binary vector definition of the letters.

21. The method of claim 20 wherein the Z different types of short k-mers in said building-block-set are characterized in that a hamming distance between each short k-mer in said building-block-set and any other short k-mer in said building-block-set is greater or equal to a certain first H1 threshold of minimal hamming distance, whereby said first threshold satisfies H1≥2; and
wherein said determining of the observed binary vector Xⁿ of dimension Z associated with location n in the data encoding sections, comprises ignoring sequenced short k-mer found at said location in one or more of the data encoding sections which does not belong to the building block set.

22. The method of claim 20 wherein said predefined alphabet Σ≡{σₘ}|_{m=1 to M} consists only of binary vectors with hamming distances between them being greater or equal to a certain second threshold H2≥2 of minimal hamming distance, thereby providing that in case said match between the observed binary vector Xⁿ and said vector of definition one of the letters {σₘ}|_{m=1 to M} of the alphabet Σ is determined, said match being indicative of validity of the reading of the encoded letter πⁿ from the locations n in said data encoding sections of sequenced molecular sequences.

23. (canceled)

24. The method of claim 20 characterized in that each letter σₘ in the alphabet letters Σ≡{σₘ}|_{m=1 to M} is defined by occurrence of a predetermined exact number Y of the different types of short k-mers of said building-block-set {E^{z}}, said predetermined exact number Y being the same for all the encoded alphabet letters πn; and wherein a stopping condition of said sequencing is that per each location n of said 1 to N locations of the data encoding sections at least said exact number Y of different types of short k-mers belonging to said building-block-set {E^{z}} is found; and wherein said sequencing is carried out at least until said stopping condition is fulfilled or until a predetermined maximal sequencing depth.

25. (canceled)

26. The method of claim 20 wherein each letter σₘ in the alphabet letters Σ≡{σₘ}_{m=1 to M}, is defined by occurrence of a predetermined and constant exact number Y of the different types of short k-mers of said building-block-set {E^{z}}, said predetermined exact number Y being the same for all the alphabet letters; and
wherein the method comprises a data reading validation/correction operation comprising selectively performing the following for each location n of said 1 to N locations of the data encoding sections at which a respective letter expected to be encoded:
(i) in case a weight Y′ of said observed binary vector Xⁿ is equal to said exact number Y, determining said encoded alphabet letters πn at the location n by mapping the observed binary vector Xⁿ to one of the alphabet letters {σₘ}|_{m=1 to M} based on a match between the observed binary vector Xⁿ and a binary vector representation of said one alphabet letter;
(ii) in case a weight Y′ of said observed binary vector Xⁿ is larger than said exact number Y, determining that an excess Y′−Y of different types of building blocks is found at the locations n of the data encoding sections; and computing statistical significances of each of the Y′ different types of building blocks found at the location n based on a number of times each of said Y′ types of building blocks is sequenced from the locations n, and:
in case statistical significance of Y′−Y types of said Y′ building blocks are below a predetermined statistical significance threshold ST, carrying out the following:
determining that said excess Y′−Y types of building blocks are the Y′−Y types of building blocks for which the statistical significance is below the threshold ST and amending said observed binary vector Xⁿ accordingly to obtain an amended observed binary vector X′ⁿ of weight Y; and
determining said encoded alphabet letters πⁿ at the location n by mapping the amended observed binary vector X′ⁿ to one of the alphabet letters {σₘ}|_{m=1 to M} based on a match between the amended observed binary vector X′ⁿ and a binary vector representation of said one alphabet letter;
in case there are less than Y′−Y types of said Y′ building blocks whose statistical significances are below the predetermined statistical significance threshold ST, determining that the observed binary vector Xⁿ may not be mapped to any one of the alphabet letters {σₘ}|_{m=1 to M} and thereby the encoded alphabet letters eat the location n is invalid;
(iii) in case a weight Y′ of said observed binary vector Xⁿ is less than said exact number Y, determining that the observed binary vector Xⁿ may not be mapped to any one of the alphabet letters {σₘ}|_{m=1 to M} and thereby the encoded alphabet letters πⁿ at the location n is invalid.

27. A data reader system adapted to implement the method according to claim 20 to read data stored in a molecular data storage system, the data reader system comprising:
a) a sequencing control module configured and operable for connecting to a sequencing system for operating the sequencing system to perform the operations (i) and (ii) of claim 20 to thereby sequence a population of molecular sequences of the data storage system; and
b) a data inference processing module configured and operable for carrying out the operation (iii) of claim 20 to determine a sequence S={πⁿ}|_{n=1 to N} of encoded letters of the alphabet Σ being inferred from the population of molecular sequences.

28. (canceled)

29. The data reader system of claim 27 wherein each letter Gm in the alphabet letters Σ≡{σₘ}|_{m=1 to M} is defined by occurrence of a predetermined exact number Y of the different types of short k-mers of said building-block-set {E^{z}}, said predetermined exact number Y being the same for all the alphabet letters; wherein a stopping condition of said sequencing is that per each location n of said 1 to N locations of the data encoding sections at least said exact number Y of different types of short k-mers belonging to said building-block-set {E^{z}} is found; and wherein said sequencing control module is adapted and to operate the sequencing system at least until said stopping condition is fulfilled or until a predetermined maximal sequencing depth.

30. The data reader system of claim 27 wherein each letter Gm in the alphabet letters Σ≡{σₘ}|_{m=1 to M}, is defined by occurrence of a predetermined exact number Y of the different types of short k-mers of said building-block-set {E^{z}}, said predetermined exact number Y being the same for all the alphabet letters; and
wherein said data inference processing module is configured and operable to carry out a data reading validation/correction operation according to the method of claim 26.

31. A method for fabricating a molecular data storage system, the method comprising:
(a) providing a support substrate having one or more spatially separated regions at which one or more respective populations of molecular sequences can be synthesized;
(b) providing one or more blocks of data to be respectively encoded by the one or more respective populations of molecular sequences which are to be synthesized at said one or more spatially separated regions respectively;
wherein said one or more blocks of data are coded by a sequence of letters S={πⁿ}|_{n=1 to N} of an alphabet Σ≡{σₘ}|_{m=1 to M};
(c) per each block of data, synthesizing a corresponding population of molecular sequences at a respective region of said one or more regions;
wherein the letters {σₘ}|_{m=1 to M} of the alphabet Σ are represented as binary occurrence vectors defined over a space spanned by Z different types of short k-mers of length k>1, which serve as data encoding molecular building blocks {Eₙ}|_{n=1 to Z} of the molecular data storage system; and
wherein said synthesizing of the population of molecular sequences at the respective region includes synthesizing the sequences of letters S={πⁿ}|_{n=1 to N} of said block of data by selectively depositing, per each letter πⁿ, all and only the data encoding building blocks indicated to be occurring by the binary vector representing the letter πⁿ.

32. The method of claim 31 wherein said depositing comprises:
(i) providing and placing said data encoding molecular building blocks indicated to be occurring by the binary vector representing the letter πⁿ and placing them at said respective region to thereby enable their binding to molecules at said region;
whereby the provided data encoding molecular building blocks are chemically “blocked” from one end thereof to prevent their binding to one another;
(ii) washing said region to remove un-bonded data encoding molecular building-blocks; and
(iii) applying un-blocking treatment to “un-block” the data encoding molecular building-blocks that are bounded to molecules at said region.

33. The method of claim 31 wherein said region of the support substrate comprises cleavable molecules adapted to bind with said data encoding molecular building-blocks, such that deposition of the basic molecular building-blocks of the first letter π¹ being encoded, are bounded to said cleavable molecules hereby enabling harvesting said population of molecules from said respective region by cleaving said cleavable molecules.

34. (canceled)

35. The method of claim 31 wherein said synthesizing of the population of molecule sequences comprises synthesizing similar population identification segments, in all molecule sequences of said population; whereby the population identification segment of each molecular sequence is indicative of the population with which the molecular sequence is associated and is different in molecular sequences of different populations.

36. A molecular data storage fabrication system adapted to implement the method according to claim 31 to fabricate a molecular data storage structure, the molecular data storage fabrication system comprising:
a container module comprising a plurality of containers including at least Z containers adapted for respectively containing Z different types of short k-mers of length k>1, being respectively data encoding molecular building-blocks serving respectively as data encoding molecular building blocks {Eₙ}|_{n=1 to Z} of the molecular data storage system;
a fabrication head fluidly connected to said Z containers and configured and operable for controlled deposition of basic molecular building-blocks contained in a one or more selected containers out of said Z containers; and
a control unit configured and operable to operate the fabrication head for implementing operations (b) and (c) of the method of claim 31; wherein said implementing comprises:
providing at least one block of data to be encoded by synthesizing a respective population of molecular sequences encoding said block of data, on a region designated for carrying said population;
wherein said at least one block of data is coded by a sequence of letters S={πⁿ}|_{n=1 to N} of an alphabet Σ≡{σₘ}|_{m=1 to M}; and wherein the letters {σₘ}|_{m=1 to M} of the alphabet Σ are represented as binary vectors (occurrence vectors) defined over a space spanned by Z different types of said data encoding molecular building blocks {Eₙ}|_{n=1 to Z};
synthesizing the population of molecular sequences encoding said block of data at the designated region, by operating said fabrication head, at said designated region to sequentially synthesize each letter πn of the sequence S; whereby for synthesize of each letter πⁿ said fabrication head selectively deposits only the molecular building blocks indicated to be occurring by the binary vector representing the letter πⁿ, from said Z containers.

37-38. (canceled)

39. The molecular data storage fabrication system of claim 36 wherein at least one of the following:
the fabrication head is configured and operable for depositing cleavable molecules at said region prior to said synthesizing; and
said molecular data storage fabrication system comprises a harvesting module configured and operable for harvesting said population of molecules from said region by cleaving said cleavable molecules.

40. (canceled)

41. The molecular data storage fabrication system of claim 36 wherein at least one of the following:
(i) said control unit is adapted for operating said fabrication head for synthesizing, for all molecules of said population, a similar identification section;
((ii) said control unit is configured and operable for operating said fabrication head to synthesize a plurality of population of molecular sequences encoding data of a plurality of respective data blocks, at different spatially separated respective regions.

42-44. (canceled)

45. A molecular label comprising the data storage system according to claim 1 and wherein said at least one data-block is being respectively encoded by the at least one population of molecular sequences.
